SEQUENCE LISTING (1 sequence)

SEQ ID NO: 1 - protein, 31 aa, Homo sapiens
HAEGTFTSDVSSYLEGQAAKEFIAWLVKGRG

(19) United States
(12) Patent Application Publication
(10) Pub. No.: US 2021/0261642 A1
(43) Pub. Date: Aug. 26, 2021

(54) SOLUBILITY OF GLP-1 PEPTIDE

(71) Applicant: Novo Nordisk A/S, Bagsvaerd (DK)

(72) Inventors: Florian Dismer, Smoerum (DK); Mattias Hansson, Lund (SE); Arne Staby, Bagsvaerd (DK)

(21) Appl. No.: 16/771,932

(22) PCT Filed: Dec. 19, 2017
(86) PCT No.: PCT/EP2017/083437

§ 371 Date: Jun. 11, 2020

(51) Int. Cl. C07K 14/605; C07K 1/02

(52) U.S. Cl. CPC C07K 14/605; C07K 1/02

(57) ABSTRACT

The present inventions relates to methods involving incubation of undissolved and/or insoluble GLP-1 peptide in solutions or suspensions comprising one or more organic solvents.

### CROSS-REFERENCE TO RELATED APPLICATIONS

This application is a 35 U.S.C. § 371 National Stage application of International Application PCT/EP2017/083437 (WO 2019/120480), filed Dec. 19, 2017; the contents of which are incorporated herein by reference.

The present invention relates to methods for increasing solubility of GLP-1 peptide.

### BACKGROUND

GLP-1 peptides exist in a first physical conformation which is physiologically active and easily dissolves in water at pH 7.4 and a second physical conformation which has very little or no GLP-1 receptor agonist activity and is substantially insoluble in water at pH 7.4. This change in physical conformation may, without being bound by any theory, be explained by conversion of alpha-helix secondary structure in the GLP-1 peptide into beta-sheet secondary structure (e.g. Kim et al, Journal of pharmaceutical sciences, 1994, 83(8), 1175-80).

Undissolved and/or insoluble GLP-1 peptide may be formed when GLP-1 solutions comprising water are agitated, exposed to hydrophobic surfaces or have large air/water interfaces. GLP-1 peptides are known to be prone to become undissolved and/or insoluble as a simple consequence of handling, for example during purification (e.g. Senderoff et al., Journal of Pharmaceutical Sciences, 1998, 87(2), 183-189). In addition, GLP-1 peptides may change into their undissolved and/or insoluble form during the process of their manufacturing. For example, mixing operations or continuous movement through a pump are common operations in large scale manufacturing processes and these operations cause the agitation, air/water interfaces and/or contact with hydrophobic surfaces that results in the undissolved and/or insoluble form of a GLP-1 peptide.

The presence of the undissolved and/or insoluble form of GLP-1 peptides greatly affects large scale production of active GLP-1 peptides. In large scale production even small amounts of undissolved and/or insoluble GLP-1 peptide decrease cost efficiency of the production.

WO01/55213 allegedly describes using very alkaline pH in aqueous solution in order to dissolve insoluble GLP-1 peptide. WO2006/051110 allegedly describes using alkaline pH in aqueous solution in combination with certain heating conditions and incubation times in order to improve physical stability of the GLP-1 peptide, etc.

Improved methods for providing GLP-1 peptides in solution are still desired, for example in order to provide high yield methods for manufacture of active, soluble GLP-1 peptide as well as stable pharmaceutical products hereof, or in order to allow simpler production methods. Such improved methods involve transforming undissolved and/or insoluble GLP-1 peptide into active, soluble GLP-1 peptide.

### SUMMARY

In some embodiments, the methods of the present invention comprise dissolving undissolved and/or insoluble GLP-1 peptides into active, soluble GLP-1 peptides in solutions or suspensions comprising water and one or more organic solvents under certain conditions.

In some embodiments the present invention relates to methods comprising the steps of: (a) obtaining a first composition comprising undissolved and/or insoluble liraglutide and the organic solvent acetonitrile or DMF, wherein said first composition is in the form of a solution or a suspension; and (b) incubating said first composition at a temperature in the range of 2-85° C., and
when said organic solvent is DMF then said incubating is carried out for a period of at least 20 minutes and (i) at a pH of less than 5.0 and at a concentration of said DMF in the range of 5-28%(w/w), or (ii) at a pH in the range of 5.0-5.5 and at a concentration of said DMF in the range of 0-35%(w/w); or
when said organic solvent is acetonitrile then said incubating is carried out for a period of at least 2 minutes and (i) at a pH of less than 6.0 and at a concentration of said acetonitrile of at least 30%(w/w), (ii) at a pH of less than 3.5 and at a concentration of said acetonitrile of at least 14%(w/w), or (iii) if said temperature is at least 50° C. then (1) at a pH in the range of 5.5-6.0 and at a concentration of said acetonitrile of less than 10% (w/w), or (2) at a pH of less than 3.5.

### Description

The present inventors surprisingly found that undissolved and/or insoluble GLP-1 peptides can be dissolved into active, soluble GLP-1 peptides in solutions comprising water and one or more organic solvents under certain conditions. In some embodiments undissolved and/or insoluble GLP-1 peptide is substantially inactive, e.g. with an EC50 on the human GLP-1 receptor of more than 100 nM, such as more than 500 nM or more than 1000 nM.

In some embodiments methods of the invention comprise the steps of (a) obtaining a composition comprising GLP-1 peptide and one or more organic solvents; and (b) incubating said solution for a period, such as a period of at least 5 minutes; wherein said composition is in the form of a solution or a suspension. In some embodiments the methods of the invention comprise the steps of (a) obtaining a composition comprising GLP-1 peptide and one or more organic solvents; and (b) incubating said solution for a period as defined herein; wherein said composition is in the form of a solution or a suspension.

In one aspect the present invention provides (x) increased amount of dissolved and/or soluble GLP-1 peptide, (y) improved storage stability of GLP-1 peptide in solution, or (z) simpler methods for providing increased amount of dissolved GLP-1 peptide. In another aspect the present invention provides a combination of one or more of (x)-(y). “Increased amount of dissolved GLP-1 peptide” may be observed as reduced amount of insoluble GLP-1 peptide and substantial identical increased in amount of soluble GLP-1 peptide. Reduction or removal of insoluble GLP-1 peptide according to the methods of this invention may be carried out in a solution comprising insoluble GLP-1 peptide, including solutions comprising both soluble and insoluble GLP-1 peptides. The invention may also solve further problems that will be apparent from the disclosure of the exemplary embodiments.

In some embodiments the methods of the invention increase the amount of dissolved and/or soluble GLP-1 peptide in a solution comprising water. In some embodiments the methods of the present invention provide increased the amount of dissolved GLP-1 peptide in a solution comprising water, such as at least 60% (w/w) water.

In some embodiments the methods of the present invention provide improved storage stability of GLP-1 peptide in solution, for example following storage at 5° C. for 1 or 2 years or following storage at room temperature for two months. In some embodiments storage stability of GLP-1 peptide as used herein refers to storage stability of GLP-1 peptide as part of an intermediate product. GLP-1 peptide as part of an intermediate product may exist in the process of preparing a pharmaceutical product, such as following a purification step. In some embodiments solubility of GLP-1 peptide as used herein refers to storage stability of GLP-1 peptide as part of an intermediate product. An intermediate product may typically be used within 2 months, such as within 2 weeks or within 1 week.

In some embodiments room temperature is 21-25° C., such as 23° C.

In some embodiments methods of the invention provide an increase of at least 0.5 mg/ml GLP-1 peptide in solution after an incubation period of 3 hours, optionally as described in Assay (I) herein. In some embodiments methods of the invention provide an increase in the amount of dissolved GLP-1 peptide of at least 0.5 mg/ml after an incubation period of 3 hours, optionally as described in Assay (I) herein.

In some embodiments the term “pH” as used herein in relation to an composition (such as a solution or a suspension) comprising an organic solvent refers to pH as determined in said composition solution without organic solvent, e.g. before addition of organic solvent.

In some embodiments methods of the invention comprise the steps of (a) obtaining a composition comprising GLP-1 peptide and one or more organic solvents; and (b) incubating said solution for a period of at least 5 minutes; wherein said composition is in the form of a solution or a suspension.

In some embodiments the method of the invention comprises the following additional step: (c) reducing the concentration of said organic solvent in the composition obtained from step (b), and optionally isolating GLP-1 peptide.

In some embodiments the incubation in step (b) of the method of the invention is carried out at a pH in the range of pH 1-13.

In some embodiments the incubation in step (b) of the method of the invention is carried out at a temperature between the freezing point and the boiling point of the solution comprising said one or more organic solvents. In some embodiments the temperature of said incubation in step (b) is above the freezing point of the solution subjected to said incubation and below 50° C. In some embodiments the temperature of said incubation in step (b) is in the range of 50-85° C.

In some embodiments the incubation in step (b) of the method of the invention is carried out for a period of 5 minutes to 48 hours.

In some embodiments the term “*” refers to multiplication. In some embodiments the term “a” means “one or more”. In some embodiments the term “about” means ±10% of the value referred to. Unless otherwise indicated in the specification, terms presented in singular form also include the plural situation.

The term “insoluble” or “substantially insoluble” when used herein in relation to GLP-1 peptide, e.g. “insoluble GLP-1 peptide”, refers to GLP-1 peptide which is inactive and which has a solubility in water at pH 7.4 of less than 0.5 mg/ml, such as less than 0.1 or 0.01 mg/ml. Similarly, the term “undissolved” when used herein in relation to GLP-1 peptide, e.g. “undissolved GLP-1 peptide”, refers to GLP-1 peptide having a solubility of less than 0.5 mg/ml, such as less than 0.1 or 0.01 mg/ml; wherein said solubility may be in a solution comprising water and/or organic solvent at any pH, e.g. at a pH in the range of pH 3-11. In some embodiments the term “undissolved” when used herein in relation to GLP-1 peptide, e.g. “undissolved GLP-1 peptide”, refers to GLP-1 peptide having a solubility in water at pH 7.4 of less than 0.5 mg/ml, such as less than 0.1 or 0.01 mg/ml.

The term “dissolved” when used herein in relation to GLP-1 peptide, e.g. “dissolved GLP-1 peptide”, refers to GLP-1 peptide having a solubility in water at pH 7.4 at least 0.5 mg/ml, such as at least 0.7 or 1 mg/ml; wherein said solubility may be in a solution comprising water and/or organic solvent at any pH, e.g. at a pH in the range of pH 3-11. In some embodiments the term “dissolved” when used herein in relation to GLP-1 peptide, e.g. “dissolved GLP-1 peptide”, refers to GLP-1 peptide having a solubility in water at pH 7.4 at least 0.5 mg/ml, such as at least 0.7 or 1 mg/ml. Similarly, the term “soluble” when used with in relation to GLP-1 peptide, e.g. “soluble GLP-1 peptide”, as used herein refers to GLP-1 peptide which is active and which has a solubility in water at pH 7.4 of at least 0.5 mg/ml, such as at least 0.7 or 1.0 mg/ml. Solubility of a GLP-1 peptide may be determined using the method of Assay (II) described herein.

The term “active” when used with in relation to GLP-1 peptide, e.g. “active GLP-1 peptide”, as used herein refers to GLP-1 peptide having a GLP-1 receptor agonist activity expressed by EC50 of below 10 nM, such as below 5 nM, below 1 nM, or below 0.5 nM. The term “inactive” when used with in relation to GLP-1 peptide, e.g. “inactive GLP-1 peptide”, as used herein refers to GLP-1 peptide having a GLP-1 receptor agonist activity expressed by EC50 of more than 20 nM, such as more than 0.1 μM, more than 0.5 μM, or more than 1 μM.

In some embodiments EC50 of the GLP-1 peptide is determined using the method of Assay (II) described herein. The term “EC50” refers to the concentration which induces a response halfway between the baseline and maximum, by reference to the dose response curve. In other words, EC50 may be seen as representing the concentration where 50% of its maximal effect is observed. The lower the EC50 value, the better the activity, also referred to as potency, of the GLP-1 peptide. In some embodiments activity of the GLP-1 peptide refers to activation of the human GLP-1 receptor. The activity of a GLP-1 peptide may be determined in a medium containing membranes expressing the human GLP-1 receptor, and/or in an assay with whole cells expressing the human GLP-1 receptor. For example, purified plasma membranes from a stable transfected cell line expressing the human GLP-1 receptor may be stimulated with the GLP-1 peptide, and the potency of cAMP production measured, e.g. based on competition between endogenously formed cAMP and exogenously added biotin-labelled cAMP, which may be captured using a specific antibody. Also, or alternatively, the response of the human GLP-1 receptor to the GLP-1 peptide may be measured in a reporter gene assay, e.g. in a stably transfected BHK cell line that expresses the human GLP-1 receptor and contains the DNA for the cAMP response element (CRE) coupled to a promoter and the gene for firefly luciferase (CRE luciferase); when cAMP is produced as a result of activation of the GLP-1 receptor this in turn results in the luciferase being expressed; luciferase may be determined by adding luciferin, which by the enzyme is converted to oxyluciferin and produces bioluminescence, which is measured and is a measure of the in vitro potency; one non-limiting example of such an assay is described in Assay (II) herein.

### Organic Solvents

The organic solvents used in the methods of the inventions are selected from the group consisting of acetonitrile, DMF and mixtures thereof. In some embodiments the organic solvent is acetonitrile. In some embodiments the organic solvent is DMF. As used herein the term “DMF” may refer to dimethylformamide.

In some embodiments the concentration of organic solvent is in the range of 0.01 to 80% (w/w). In some embodiments the concentration of organic solvent is less than the concentration at which no more than 0.5 mg/ml of the GLP-1 peptide in solution precipitates within 3 hours at room temperature. In some embodiments the organic solvent is phenol at a concentration of at least 1%(w/w). In some embodiments the term “concentration of organic solvent” refers to the total concentration of organic solvent in case of the presence of more than one organic solvent. In some embodiments the term “concentration of organic solvent” refers to concentration of organic solvent determined before the first incubation of the present invention, e.g. after step (a) and before step (b).

### End Product

In some embodiments the composition obtained from step (b) is in the form of a solution. In some embodiments the composition obtained from step (b) is in the form of a suspension. In some embodiments undissolved GLP-1 peptide in the composition obtained from step (b) of the method of the present invention is removed from said composition, for example by filtration. In some embodiments the concentration of organic solvent in the composition obtained from step (b) is reduced, e.g. by dilution, chromatography, or UF/DF filtration.

### Starting Material

The composition comprising GLP-1 peptide comprises water and may be in the form of a solution or a suspension. In some embodiments the solution or suspension comprising GLP-1 peptide may be prepared from GLP-1 peptide in solid form, such as a precipitate or powder, for example prepared by Method 2 of Assay (I) as described herein. The powder may be formed by evaporation of the solvent from a solution or suspension, for example prepared by Method 1 of Assay (I) as described herein. The suspension may comprise GLP-1 peptide in solid form. In some embodiments the suspension comprises GLP-1 peptide which is insoluble.

The solution or suspension comprising GLP-1 peptide may be prepared by adding solvent, such as water and/or organic solvent, to a GLP-1 peptide composition. The solution or suspension comprising GLP-1 peptide may be prepared as described in the examples herein.

### Particular Method Steps

### Liraglutide

In some embodiments the method of the invention comprises incubation of a composition comprising liraglutide according to any one of the Equations (Eq.) 1-92 with solubility criteria as defined in Tables 1 a, 1 b and 2. These Equations may also simply be referred to herein as “Equations as defined in Tables 1 a, 1b and 2” or with reference to a specific Equation, e.g. “Equation 1” for Eq. 1 in these tables.

TABLE 1a
Parameters for equation: y = p1*x^{6 +} p2*x⁵ + p3*x⁴ +
p4*x³ + p5*x² + p6*x + p7 defining soluble areas for each condition shown in Table 2.

| | Parameter | | | |
|---|---|---|---|---|
| Eq. | p1 | p2 | p3 | p4 |
| 32 | 3.76823717E−08 | −4.29553097E−06 | 1.84781739E−04 | −3.69602295E−03 |
| 33 | 2.39824330E−10 | −5.20504520E−08 | 3.36818410E−06 | 2.74075690E−06 |
| 34 | −7.25474407E−10 | 1.70934793E−07 | −1.53430846E−05 | 6.43323233E−04 |
| 35 | −3.42985250E−09 | 9.31342080E−07 | −1.03502170E−04 | 6.02276220E−03 |
| 36 | 0 | 0 | 0 | 7.49654506E−06 |
| 37 | −7.00430900E−08 | 1.52685100E−05 | −1.36028810E−03 | 6.33540860E−02 |
| 65 | 0 | −2.74062507E−07 | 4.99594283E−05 | −3.31793309E−03 |
| 66 | 3.58646550E−08 | −8.88273820E−06 | 8.85078870E−04 | −4.51054080E−02 |
| 67 | | 1.99478105E−07 | −2.66958963E−05 | 1.25984196E−03 |
| 68 | −6.17051210E−09 | 1.59042550E−06 | −1.63576160E−04 | 8.54340630E−03 |
| 69 | | 4.44640975E−08 | −5.08367791E−06 | 1.50515218E−04 |
| 70 | 1.07785200E−08 | −1.24783720E−06 | −1.71991140E−04 | 3.89468020E−02 |
| 71 | 0 | 0 | 0 | 0 |
| 72 | 0 | 9.31179132E−09 | −2.39306155E−06 | 2.26503774E−04 |
| 73 | −2.68430400E−08 | 5.65396200E−06 | −4.88876830E−04 | 2.21509860E−02 |
| 74 | 0 | 4.48714216E−09 | 2.83486304E−06 | −3.56587344E−04 |
| 75 | −2.61280320E−09 | 7.15417820E−07 | −7.93210930E−05 | 4.49744770E−03 |
| 76 | 3.04932566E−09 | −6.24903964E−07 | 4.90088677E−05 | −1.85930823E−03 |
| 77 | 1.58051050E−09 | −2.67024330E−07 | 8.30370210E−06 | 8.12230720E−04 |
| 78 | −4.11211452E−09 | 4.85865425E−07 | −1.29165606E−05 | −3.70711516E−04 |
| 79 | −4.68618550E−09 | 7.99910650E−07 | −5.01964260E−05 | 1.43813850E−03 |
| 80 | 3.18984260E−02 | −1.40850260E+00 | 2.53491200E+01 | −2.37637010E+02 |
| 81 | −2.51443877E−09 | 2.39202492E−07 | −6.80130641E−06 | −1.14586983E−04 |
| 82 | 4.83732320E−09 | −4.01375240E−07 | 1.02596980E−05 | −4.47337410E−05 |
| 83 | 0 | 0 | 0 | 0 |
| 84 | 0 | −1.54250067E−08 | 3.88982883E−06 | −3.68886274E−04 |
| 85 | 0 | 1.00042090E−07 | −1.55239400E−05 | 7.42151640E−04 |
| 86 | 0 | −5.58456840E−08 | 9.01266920E−06 | −5.06724750E−04 |
| 87 | 0 | 6.33452710E−08 | −6.08477920E−06 | 2.71297030E−04 |

E: multiplied by 10 to the power of, for example “E−03” means “*10⁻³”.

TABLE 1b
Parameters for equation: y = p1*x^{6 +} p2*x⁵ + p3*x⁴ +
p4*x³ + p5*x² + p6*x + p7 defining soluble areas for each condition shown in Table 2.

| | Parameters for equation: y = p1*x^{6 +}p2*x⁵ + p3*x⁴ + p4*x³ + p5x² + p6*x + p7 | | |
|---|---|---|---|
| Eq. | p5 | p6 | p7 |
| 32 | 3.39466168E−02 | −1.24847077E−01 | 6.50568384E+00 |
| 33 | −8.09488750E−03 | 2.87376310E−01 | 3.75182950E−01 |
| 34 | −1.20092757E−02 | 6.55767799E−02 | 6.42705362E+00 |
| 35 | −1.93047830E−01 | 3.21683160E+00 | −1.81601070E+01 |
| 36 | −6.55584965E−04 | 1.16126759E−02 | 6.41116827E+00 |
| 37 | −1.62674830E+00 | 2.18374180E+01 | −1.16250270E+02 |
| 65 | 1.02349676E−01 | −1.51947349E+00 | 1.49571064E+01 |
| 66 | 1.22777560E+00 | −1.66580550E+01 | 8.85260440E+01 |
| 67 | −2.48726570E−02 | 1.55062531E−01 | 6.40213794E+00 |
| 68 | −2.38084960E−01 | 3.36715470E+00 | −1.57064940E+01 |
| 69 | −2.21541800E−04 | −2.89108331E−02 | 6.49017991E+00 |
| 70 | −2.70566480E+00 | 8.32911320E+01 | −9.68492950E+02 |
| 71 | 1.31200000E+01 | −2.68900000E+02 | 1.44200000E+03 |
| 72 | −4.92364937E−03 | −8.75968776E−02 | 7.81395261E+00 |
| 73 | −5.50849580E−01 | 7.04029350E+00 | −3.25708830E+01 |
| 74 | 1.32932556E−02 | −1.82504997E−01 | 6.53035245E+00 |
| 75 | −1.34820000E−01 | 1.97994740E+00 | −7.85450440E+00 |
| 76 | 3.54276298E−02 | −2.86180548E−01 | 6.58053221E+00 |
| 77 | −6.11558620E−02 | 1.34640240E+00 | −6.28223490E+00 |
| 78 | 1.97615076E−02 | −2.19855864E−01 | 6.06043350E+00 |
| 79 | −1.87842920E−02 | 9.49511420E−02 | 3.39265440E+00 |
| 80 | 1.22169200E+03 | −3.25827290E+03 | 3.57904430E+03 |
| 81 | 6.37855786E−03 | −2.17048515E−03 | 5.08251579E+00 |
| 82 | −1.37099980E−03 | 1.15233330E−02 | 3.48585500E+00 |
| 83 | 0 | 0 | 4.00000000E+01 |
| 84 | 1.55688595E−02 | −1.83126095E−01 | 6.49627535E+00 |
| 85 | −8.91519960E−03 | −4.18911940E−02 | 5.31782360E+00 |
| 86 | 1.26292740E−02 | −1.14338300E−01 | 5.30973960E+00 |
| 87 | −9.97372280E−03 | 1.86951180E−01 | 2.85761730E+00 |

E: multiplied by 10 to the power of, for example “E−03” means “*10⁻³”.

TABLE 2
Treatment conditions and soluble areas defined by equations in Tables 1a and 1b.

| | | Conditions | | | | Boundaries | | Parameters | | Soluble area |
|---|---|---|---|---|---|---|---|---|---|---|
| Eq. | Mode | Modifier | Temp. | Time | | pH | Org. | x | y | criterion |
| 32 | Pr | MeCN | 5° C. | 5 | min | >5.0 | | Org. | pH | y > calculated |
| 33 | Pr | | | | | <5.0 | | Org. | pH | y < calculated |
| 34 | Pr | MeCN | 5° C. | 180 | min | >5.0 | | Org. | pH | y > calculated |
| 35 | Pr | | | | | <5.0 | | Org. | pH | y < calculated |
| 36 | Pr | MeCN | 5° C. | 24 | h | >5.0 | | Org. | pH | y > calculated |
| 37 | Pr | | | | | <5.0 | | Org. | pH | y < calculated |
| 65 | Ev | MeCN | 5° C. | 5 | min | >5.0 | | Org. | pH | y > calculated |
| 66 | Ev | | | | | <5.0 | 20.0 < × < 60.0 | Org. | pH | y < calculated |
| 67 | Ev | MeCN | 5° C. | 180 | min | >4.5 | | Org. | pH | y > calculated |
| 68 | Ev | | | | | <4.5 | | Org. | pH | y < calculated |
| 69 | Ev | MeCN | 5° C. | 24 | h | 5.0 < 8.0 | | Org. | pH | y > calculated |
| 70 | Ev | | | | | <5.0 | | Org. | pH | y < calculated |
| 71 | Ev | | | | | >8.0 | | pH | Org. | y < calculated |
| 72 | Ev | MeCN | RT | 5 | min | >4.5 | | Org. | pH | y > calculated |
| 73 | Ev | | | | | <4.5 | | Org. | pH | y < calculated |
| 74 | Ev | MeCN | RT | 180 | min | >4.5 | | Org. | pH | y > calculated |
| 75 | Ev | | | | | <4.5 | | Org. | pH | y < calculated |
| 76 | Ev | MeCN | RT | 24 | h | >5.0 | | Org. | pH | y > calculated |
| 77 | Ev | | | | | <5.0 | | Org. | pH | y < calculated |
| 78 | Ev | MeCN | 70° C. | 5 | min | >4.6 | <54.5 | Org. | pH | y > calculated |
| 79 | Ev | | | | | <4.6 | <54.5 | Org. | pH | y < calculated |
| 80 | Ev | | | | | 3.0-11.0 | >54.5 | pH | Org. | y < calculated |
| 81 | Ev | MeCN | 70° C. | 180 | min | >4.3 | <40.0 | Org. | pH | y > calculated |
| 82 | Ev | | | | | <4.3 | <40.0 | Org. | pH | y < calculated |
| 83 | Ev | | | | | | >40.0 | pH | Org. | y > calculated |
| 84 | Ev | DMF | RT | 5 | min | 3.0-11.0 | 0-70 | Org. | pH | y > calculated |
| 85 | Ev | DMF | RT | 180 | min | 3.0-11.0 | 0-70 | Org. | pH | y > calculated |
| 86 | Ev | DMF | RT | 24 | h | >4.5 | 0-70 | Org. | pH | y > calculated |
| 87 | Ev | | | | | <4.5 | <40 | Org. | pH | y < calculated |

Pr: Plate prepared by precipitation.
Ev: Plate prepared by evaporation.
RT: Room temperature.
Modifier: Organic solvent.
Org.: Concentration of organic solvent (% w/w).
ISOP: Isopropanol. As an example, “y < calculated” means that pH should be less than the pH calculated according to the equation defined in Tables 1a, 1b, and 2.

In some embodiments pH and concentration of organic solvent is as defined by Equation 32, 33, 34 as defined herein. In some embodiments pH and concentration of organic solvent is as defined by Equation 35, 36, 37 as defined herein. In some embodiments pH and concentration of organic solvent is as defined by Equation 65, 66, 67 as defined herein. In some embodiments pH and concentration of organic solvent is as defined by Equation 68, 69, or 70 as defined herein. In some embodiments pH and concentration of organic solvent is as defined by Equation 71, 72, or 73 as defined herein. In some embodiments pH and concentration of organic solvent is as defined by Equation 74, 75, or 76 as defined herein. In some embodiments pH and concentration of organic solvent is as defined by Equation 77, 78, or 79 as defined herein. In some embodiments pH and concentration of organic solvent is as defined by Equation 80, 81, 82, or 83 as defined herein. In some embodiments pH and concentration of organic solvent is as defined by Equation 84, 85, 86, or 87 as defined herein.

In some embodiments, when the organic solvent is acetonitrile; then the incubating step (b) is (i) at a pH of less than 6.0 and at a concentration of said acetonitrile of at least 30% (w/w); (ii) at a pH of less than 3.5 and at a concentration of said acetonitrile of at least 14%(w/w); or (iii) if said temperature is at least 40° C. then (1) at a pH in the range of 5.5-6.0 and at a concentration of said acetonitrile of less than 10%(w/w), or (2) at a pH of less than 3.5. In some embodiments the invention relates to a method comprising the steps of: (a) obtaining a first composition comprising undissolved and/or insoluble liraglutide and the organic solvent acetonitrile, wherein said first composition is in the form of a solution or a suspension; and (b) incubating said first composition for a period of at least 2 minutes and at a temperature in the range of 2-85° C.; and the incubation is carried out (i) at a pH of less than 6.0 and at a concentration of said acetonitrile of at least 30%(w/w); (ii) at a pH of less than 3.5 and at a concentration of said acetonitrile of at least 14%(w/w); or (iii) if said temperature is at least 40° C. then (1) at a pH in the range of 5.5-6.0 and at a concentration of said acetonitrile of less than 10%(w/w), or (2) at a pH of less than 3.5. In step (b)(i) the pH may be at least 3.5. In step (b)(i) the concentration of said acetonitrile may be in the range of 30-100%(w/w) or at least 40%(w/w). In step (b)(ii) the concentration of said acetonitrile may be at least 18%(w/w) or at least 20%(w/w). In step (b)(ii) the concentration of said acetonitrile may be at least 30%(w/w), such as at least 33%(w/w) or at least 35%(w/w). In step (b)(ii) the concentration of said acetonitrile may be at least 44%(w/w), such as at least 45%(w/w) or at least 46%(w/w). In step (b)(ii) the concentration of said acetonitrile may be at least 55%(w/w). In step (b)(ii) the concentration of said acetonitrile may be up to 98%(w/w), such as up to 95%(w/w) or up to 90%(w/w). In step (b)(ii) the concentration of said acetonitrile may be up to 80%(w/w), such as up to 70%(w/w) or up to 60%(w/w). In step (b)(ii) the concentration of said acetonitrile may be up to 60%(w/w), such as up to 40%(w/w) or up to 22%(w/w). In step (b)(ii) the concentration of said acetonitrile may be 30-100%(w/w) or at least 40%(w/w). In step (b)(ii) said pH may be less than 3.4, less than 3.3 or less than 3.2. In step (b)(ii) said pH may be at least 1.0, at least 1.5, or at least 2.0. In step (b)(ii) said pH may be at least 2.5, or at least 3.0. The temperature of said incubation in step (b)(i) may be selected from the group consisting of 2-40° C., 40-85° C. or 10-50° C. In step (b)(i) said temperature may be in the range of 5-50° C. or in the range of 10-40° C. In step (b)(i) said temperature may be in the range of in the range of 15-30° C. or in the range of 21-25° C. In step (b)(i) said period may be in the range of 5 min to 2 hours, such as in the range of 5-30 min, or in the range of 2-6 hours. In step (b)(i) said period may be in the range of 1-48 hours or in the range of 8-40 hours. In step (b)(i) said period may be in the range of 12-36 hours or in the range of 18-32 hours. In step (b)(iii)(2) the concentration of said acetonitrile may be 0-20%(w/w) or at least 28%(w/w), such as 30-100%(w/w). In step (b)(iii)(2) the concentration of said acetonitrile may be at least 40%(w/w) or at least 42%(w/w). In step (b)(iii)(2) the concentration of said acetonitrile may be at least 45%(w/w) or at least 50%(w/w). In step (b)(iii)(2) the concentration of said acetonitrile may be 1-18%(w/w) or 2-16%(w/w). In step (b)(ii) said temperature may be in the range of 2-40° C., such as at least 5-30° C. In step (b)(ii) said concentration of acetonitrile may be in the range of 15-100%(w/w). In step (b)(ii) said concentration of acetonitrile may be at least 15%(w/w), such as at least 18%(w/w) or at least 20%(w/w). In step (b)(ii) said concentration of acetonitrile may be no more than 99% (w/w), such as no more than 98%(w/w) or no more than 95%(w/w). In step (b)(ii) said concentration of acetonitrile may be no more than 55%(w/w), such as no more than 40% (w/w) or no more than 22%(w/w). In step (b)(ii) said pH may be in the range of 1-3.5. In step (b)(ii) or in step (b)(iii)(2) said pH may be less than 3.4 or less than 3.3. In step (b)(ii) or in step (b)(iii)(2) said pH may be less than 3.2, such as less than 3.1 or less than 3.0. In step (b)(i) or step (b)(iii), such as step (b)(iii)(1) or step (b)(iii)(2), said temperature may be at least 40° C., such as at least 50° C. or at least 60° C.

### DMF

In some embodiments, when the organic solvent is DMF; the incubating step (b) is for a period of at least 20 minutes and (i) at a pH of less than 5.0 and at a concentration of said DMF in the range of 5-28%(w/w); or (ii) at a pH in the range of 5.0-5.5 and at a concentration of said DMF in the range of 0-35%(w/w). In some embodiments the invention relates to a method comprising the steps of: (a) obtaining a first composition comprising undissolved and/or insoluble liraglutide and the organic solvent DMF, wherein said first composition is in the form of a solution or a suspension; and (b) incubating said first composition for a period of at least 20 minutes and at a temperature in the range of 2-85° C.; and the incubation is carried out (i) at a pH of less than 5.0 and at a concentration of said DMF in the range of 5-28%(w/w); or (ii) at a pH in the range of 5.0-5.5 and at a concentration of said DMF in the range of 0-35%(w/w). In step (b)(i) said pH may be less than 4 and optionally at least 1. In step (b)(i) said pH may be less than 3.8, such as less than 3.7 or less than 3.6. In step (b)(i) the concentration of said DMF may be in the range of 7-23%(w/w) or in the range of 8-26%(w/w). In step (b)(i) the concentration of said DMF may be in the range of 9-25%(w/w) or in the range of 10-23%(w/w). In step (b)(i) said temperature may be in the range of 5-50° C. or in the range of 10-40° C. In step (b)(i) said temperature may be in the range of in the range of 15-30° C. or in the range of 21-25° C. In step (b)(i) said period may be in the range of 1-48 hours or in the range of 8-40 hours. In step (b)(i) said period may be in the range of 12-36 hours or in the range of 18-32 hours.The method according to any one of the preceding embodiments, wherein said organic solvent may be DMF, and in step (b)(i) the concentration of said DMF may be in the range of 7-23%(w/w). The method according to the preceding embodiment, wherein said period may be at least 1 hour.The method according to any one of the preceding embodiments, wherein said organic solvent may be DMF, and in step (b)(i) a. said pH may be less than 3.8, b. the concentration of said DMF may be in the range of 9-25%(w/w), and c. said period may be at least 8 hours. In step (b)(i) the concentration of said DMF may be at least 8%(w/w) or at least 10%(w/w). In step (b)(i) the concentration of said DMF may be up to 22%(w/w), up to 21%(w/w) or up to 20%(w/w). In step (b)(i) the pH may be in the range of from 1.0 to less than 5.0. In step (b)(ii) the pH may be at least 2.0, at least 2.5 or at least 3.0. In step (b)(ii) the pH may be less than 4.8 or less than 4.9. In step (b)(ii) the pH may be in the range of 4.8-6.0. In step (b)(ii) the pH may be at least 4.8 or at least 4.9. In step (b)(ii) the pH may be up to 5.8, up to 5.7, or up to 5.6. In step (b)(ii) the pH may be up to 6.0 or up to 5.9. In step (b)(ii) the concentration of said DMF may be at least 1%(w/w), at least 2%(w/w), or at least 3%(w/w). In step (b)(ii) the concentration of said DMF may be at least 1%(w/w), at least 4%(w/w), or at least 5%(w/w). In step (b)(ii) the concentration of said DMF may be up to 37%(w/w), up to 32%(w/w) or up to 30%(w/w). In step (b) the incubation time may be less than 8 hours. In step (b) the incubation time may be less than 6 hours. In step (b) the incubation time may be less than 5 hours. In step (b) the incubation time may be in the range from 30 min to less than 8 hours. In step (b) the incubation time may be at least 1 hour. In step (b) the incubation time may be at least 1.5 hours. In step (b) if the incubation time may be at least 8 hours, then the incubation may be carried out (i) at a pH of less than 5.0 and at a concentration of said DMF in the range of 2-40%(w/w); or (ii) at a pH in the range of 5.0-6.0 and at a concentration of said DMF in the range of 0-40%(w/w). In step (b)(i) said DMF may be at a concentration at least 3%(w/w), at least 4%(w/w), or at least 5%(w/w). In step (b)(i) said DMF may be at a concentration up to 35%(w/w), up to 30%(w/w), or up to 28%(w/w). In step (b)(i) said DMF may be at a concentration up to 27%(w/w), up to 26%(w/w), or up to 25%(w/w). In step (b)(i) said pH may be less than 4.5, less than 4.4 or less than 4.3. In step (b)(i) said pH may be less than 4.2, less than 4.1 or less than 4.0. In step (b)(i) said pH may be less than 3.9, less than 3.8 or less than 3.7. In step (b)(i) said pH may be at least 1.0, at least 1.5, or at least 2.0. In step (b)(i) said pH may be at least 2.5, or at least 3.0. In step (b)(ii) if pH may be less than 5.5 then the concentration of said DMF may be in the range of 0-20%(w/w). In step (b)(ii) if pH may be less than 5.4 or less than 5.3 then the concentration of said DMF may be in the range of 0-20%(w/w). In step (b)(ii) the concentration of said DMF may be up to 20%(w/w) or up to 19%(w/w). In step (b)(ii) the concentration of said DMF may be up to 18%(w/w) or up to 17%(w/w).

In some embodiments the method comprises obtaining a composition comprising substantially no acetonitrile or DMF.

### GLP-1 Peptide

The term “GLP-1 peptide” as used herein refers to a compound comprising a peptide and which, when active, fully or partially activates the human GLP-1 receptor. In some embodiments the GLP-1 peptide is a GLP-1 analogue, optionally comprising one substituent. The term “analogue” as used herein referring to a GLP-1 peptide (hereafter “peptide”) means a peptide wherein at least one amino acid residue of the peptide has been substituted with another amino acid residue and/or wherein at least one amino acid residue has been deleted from the peptide and/or wherein at least one amino acid residue has been added to the peptide and/or wherein at least one amino acid residue of the peptide has been modified. Such addition or deletion of amino acid residues may take place at the N-terminal of the peptide and/or at the C-terminal of the peptide. In some embodiments a simple nomenclature is used to describe the GLP-1 peptide, e.g., [Aib8] GLP-1(7-37) designates an analogue of GLP-1(7-37) wherein the naturally occurring Ala in position 8 has been substituted with Aib. In some embodiments the GLP-1 peptide comprises a maximum of twelve, such as a maximum of 10, 8 or 6, amino acids which have been alterered, e.g., by substitution, deletion, insertion and/or modification, compared to e.g. GLP-1(7-37). In some embodiments the analogue comprises up to 10 substitutions, deletions, additions and/or insertions, such as up to 9 substitutions, deletions, additions and/or insertions, up to 8 substitutions, deletions, additions and/or insertions, up to 7 substitutions, deletions, additions and/or insertions, up to 6 substitutions, deletions, additions and/or insertions, up to 5 substitutions, deletions, additions and/or insertions, up to 4 substitutions, deletions, additions and/or insertions or up to 3 substitutions, deletions, additions and/or insertions, compared to e.g. GLP-1(7-37). Unless otherwise stated the GLP-1 comprises only L-amino acids.

In some embodiments the term “GLP-1 analogue” or “analogue of GLP-1” as used herein refers to a peptide, or a compound, which is a variant of the human Glucagon-Like Peptide-1 (GLP-1(7-37)). GLP-1(7-37) has the sequence HAEGTFTSDV SSYLEGQAAKEFIAWLVKGRG (SEQ ID No: 1). In some embodiments the term “variant” refers to a compound which comprises one or more amino acid substitutions, deletions, additions and/or insertions.

In some embodiments the GLP-1 peptide exhibits at least 60%, 65%, 70%, 80% or 90% sequence identity to GLP-1(7-37) over the entire length of GLP-1(7-37). As an example of a method for determination of sequence identity between two analogues the two peptides [Aib8]GLP-1(7-37) and GLP-1(7-37) are aligned. The sequence identity of [Aib8]GLP-1(7-37) relative to GLP-1(7-37) is given by the number of aligned identical residues minus the number of different residues divided by the total number of residues in GLP-1(7-37). Accordingly, in said example the sequence identity is (31-1)/31.

The concentration of GLP-1 peptide may be determined using any suitable method. For example, LC-MS (Liquid Chromatography Mass Spectroscopy) may be used, or immunoassays such as RIA (Radio Immuno Assay), ELISA (Enzyme-Linked Immuno Sorbent Assay), and LOCI (Luminescence Oxygen Channeling lmmunoasssay). General protocols for suitable RIA and ELISA assays are found in, e.g., WO 2009/030738 on p. 116-118.

In some embodiments the GLP-1 peptide is liraglutide. Liraglutide is Arg34,Lys26-(N-epsilon-(gamma-L-glutamyl(N-alfa-hexadecanoyl)))-GLP-1(7-37), also known as N²⁶-(hexadecanoyl-γ-glutamyle)-[34-arginine]GLP-1-(7-37)-peptide (WHO Drug Information Vol. 17, No. 2, 2003). Liraglutide may be prepared as described in Example 37 of WO98/08871.

Processes for production GLP-1 peptides are well-known in the art. The amino acid sequence of the GLP-1 peptide (or fragments thereof), for example the unbranched amino acid sequence such as Arg34-GLP-1(7-37), may for instance be produced by classical peptide synthesis, e.g., solid phase peptide synthesis using t-Boc or Fmoc chemistry or other well established techniques, see, e.g., Greene and Wuts, “Protective Groups in Organic Synthesis”, John Wiley & Sons, 1999, Florencio Zaragoza Dorwald, “Organic Synthesis on solid Phase”, Wiley-VCH Verlag GmbH, 2000, and “Fmoc Solid Phase Peptide Synthesis”, Edited by W.C. Chan and P.D. White, Oxford University Press, 2000. Also, or alternatively, they may be produced by recombinant methods, viz. by culturing a host cell containing a DNA sequence encoding the unbranched amino acid sequence (peptide) and capable of expressing the peptide in a suitable nutrient medium under conditions permitting the expression of the peptide. Non-limiting examples of host cells suitable for expression of these peptides are: Escherichia coli, Saccharomyces cerevisiae, as well as mammalian BHK or CHO cell lines.

The GLP-1 peptide may be in the form of a pharmaceutically acceptable salt, amide, or ester. Salts are e.g. formed by a chemical reaction between a base and an acid, e.g.: 2NH₃+H₂SO₄→(NH4)2504. The salt may be a basic salt, an acid salt, or it may be neither nor (i.e. a neutral salt). Basic salts produce hydroxide ions and acid salts hydronium ions in water. The salts of GLP-1 peptide may be formed with added cations or anions between anionic or cationic groups, respectively. These groups may be situated in the peptide moiety, and/or in the side chain of GLP-1 peptide. Arg34-GLP-1(7-37) may be regarded as the peptide moiety of GLP-1 peptide. Gamma-L-glutamyl(N-alfa-hexadecanoyl) may be regarded as the side chain of GLP-1 peptide. Non-limiting examples of anionic groups of GLP-1 peptide include free carboxylic groups in the side chain as well as in the peptide moiety. The peptide moiety of GLP-1 peptide includes a free carboxylic acid group at the C-terminus, and it may also include free carboxylic groups at internal acid amino acid residues, such as Asp and Glu. Non-limiting examples of cationic groups in the peptide moiety include the free amino group at the N-terminus as well as any free amino group of internal basic amino acid residues, such as His, Arg, and Lys. The ester of GLP-1 peptide may be formed by the reaction of a free carboxylic acid group with an alcohol or a phenol, which leads to replacement of at least one hydroxyl group by an alkoxy or aryloxy group. The ester formation may involve the free carboxylic group at the C-terminus of the peptide, and/or any free carboxylic group in the side chain. The amide of GLP-1 peptide may be formed by the reaction of a free carboxylic acid group with an amine or a substituted amine, or by reaction of a free or substituted amino group with a carboxylic acid. The amide formation may involve the free carboxylic group at the C-terminus of the peptide, any free carboxylic group in the side chain, the free amino group at the N-terminus of the peptide, and/or any free or substituted amino group of the peptide in the peptide and/or the side chain. In some embodiments GLP-1 peptide is in the form of a pharmaceutically acceptable salt. In some embodiments GLP-1 peptide is in the form of a pharmaceutically acceptable amide, preferably with an amide group at the C-terminus of the peptide. In some embodiment GLP-1 peptide is in the form a pharmaceutically acceptable ester.

### Pharmaceutical Compositions

The product obtained from the methods of the present invention may be a pharmaceutical composition or an intermediate composition used in the manufacture of a pharmaceutical composition. The pharmaceutical composition may comprise one or more pharmaceutically acceptable excipients. The term “excipient” broadly refers to any component other than the active therapeutic ingredient(s). The excipient may be an inert substance, an inactive substance, and/or a not medicinally active substance. The excipient may serve various purposes, e.g. as a carrier, vehicle, diluent, tablet aid, and/or to improve administration, and/or absorption of the active substance. The formulation of pharmaceutically active ingredients with various excipients is known in the art, see e.g. Remington: The Science and Practice of Pharmacy (e.g. 19th edition (1995), and any later editions). Non-limiting examples of excipients are: Solvents, diluents, buffers, preservatives, tonicity regulating agents, chelating agents, and stabilisers. The pharmaceutical composition may have a pH in the range of 7.0-10.0, such as 7.4-9.0 or 7.8-8.4. In some embodiments pH of said pharmaceutical composition is in the range of 8.0-8.3, e.g. 8.15. In some embodiments said excipients are one or more selected from the group consisting of isotonic agent (e.g. propylene glycol), buffer (e.g. phosphate buffer, such as disodium phosphate dihydrate), and a preservative (e.g. phenol).

In some embodiments the methods of the present invention provides a stable pharmaceutical composition. The term “stable pharmaceutical composition” when used herein refers to a composition, e.g. a solution or suspension, comprising GLP-1 peptide, and which composition following storage at least 90%(w/w) of said GLP-1 peptide remains in solution in said composition. The conditions of storage for this stable pharmaceutical composition may be at 5° C. for 1 or 2 years. Alternatively, the conditions of this storage may be at 5° C. for 24 hours or 1 week. In yet another alternative, the conditions of this storage may room temperature for two months. The ratio of GLP-1 peptide remaining in solution in said stable pharmaceutical composition may be at least 95%(w/w), such as at least 97%(w/w) or at least 99%(w/w).

### Indications

The pharmaceutical composition obtained by the methods of the invention may be for use in medicine. The pharmaceutical composition of the invention may be for use in the treatment and/or prevention of type 2 diabetes or obesity. In some embodiments the pharmaceutical composition of the invention is for use in prevention and/or treatment of diabetic complications, such as angiopathy; neuropathy, including peripheral neuropathy; nephropathy; and/or retinopathy. In some embodiments the pharmaceutical composition of the invention is for use in prevention and/or treatment of one or more cardiovascular diseases. In some embodiments the pharmaceutical composition of the invention is for use in prevention and/or treatment of sleep apnoea.

### Non-Limiting Particular Embodiments of the Invention

1. A method comprising the steps of:
(a) obtaining a first composition comprising undissolved and/or insoluble GLP-1 peptide and one or more organic solvents selected from the group consisting of acetonitrile and DMF, wherein said first composition is in the form of a solution or a suspension; and
(b) incubating said first composition (e.g. solution) for a period of at least 2 minutes at a temperature in the range of 2-85° C.; with the proviso that if GLP-1 peptide is liraglutide and the organic solvent is phenol then the concentration of said organic solvent is at least 1%(w/w); wherein said method increases the amount of dissolved and/or insoluble GLP-1 peptide.
2. A method comprising the steps of:
(a) obtaining a first composition comprising undissolved and/or insoluble liraglutide and the organic solvent acetonitrile or DMF, wherein said first composition is in the form of a solution or a suspension; and
(b) incubating said first composition at a temperature in the range of 2-85° C., and
   when said organic solvent is DMF then said incubating is carried out for a period of at least 20 minutes and (i) at a pH of less than 5.0 and at a concentration of said DMF in the range of 7-23%(w/w), or (ii) at a pH in the range of 5.0-5.5 and at a concentration of said DMF in the range of 0-35%(w/w); or
   when the organic solvent is acetonitrile then said incubating is carried out for a period of at least 2 minutes and (i) at a pH of less than 6.0 and at a concentration of said acetonitrile of at least 30%(w/w), (ii) at a pH of less than 3.5 and at a concentration of said acetonitrile of at least 14%(w/w), or (iii) if said temperature is at least 50° C. then (1) at a pH in the range of 5.5-6.0 and at a concentration of said acetonitrile of less than 10%(w/w), or (2) at a pH of less than 3.5.
3. A method comprising the steps of:
(a) obtaining a first composition comprising undissolved and/or insoluble liraglutide and the organic solvent acetonitrile or DMF, wherein said first composition is in the form of a solution or a suspension; and
(b) incubating said first composition at a temperature in the range of 2-85° C., and
   when said organic solvent is DMF then said incubating is carried out for a period of at least 20 minutes and (i) at a pH of less than 5.0 and at a concentration of said DMF in the range of 7-23%(w/w), or (ii) at a pH in the range of 5.0-5.5 and at a concentration of said DMF in the range of 0-35%(w/w); or
   when said organic solvent is acetonitrile then said incubating is carried out for a period of at least 2 minutes and (i) at a pH of less than 6.0 and at a concentration of said acetonitrile of at least 30%(w/w), (ii) at a pH of less than 3.5 and at a concentration of said acetonitrile of at least 14%(w/w), or (iii) if said temperature is at least 50° C. then (1) at a pH in the range of 5.5-6.0 and at a concentration of said acetonitrile of less than 10%(w/w), or (2) at a pH of less than 3.5.
4. The method according to any one of the preceding embodiments, wherein the organic solvent is acetonitrile; and said incubating step (b) is
   (i) at a pH of less than 6.0 and at a concentration of said acetonitrile of at least 30%(w/w);
   (ii) at a pH of less than 3.5 and at a concentration of said acetonitrile of at least 14%(w/w); or
   (iii) if said temperature is at least 40° C. then (1) at a pH in the range of 5.5-6.0 and at a concentration of said acetonitrile of less than 10%(w/w), or (2) at a pH of less than 3.5.
5. A method comprising the steps of:
(a) obtaining a first composition comprising undissolved and/or insoluble liraglutide and the organic solvent acetonitrile, wherein said first composition is in the form of a solution or a suspension; and
(b) incubating said first composition for a period of at least 2 minutes and at a temperature in the range of 2-85° C.; and the incubation is carried out
   (i) at a pH of less than 6.0 and at a concentration of said acetonitrile of at least 30%(w/w);
   (ii) at a pH of less than 3.5 and at a concentration of said acetonitrile of at least 14%(w/w); or
   (iii) if said temperature is at least 40° C. then (1) at a pH in the range of 5.5-6.0 and at a concentration of said acetonitrile of less than 10%(w/w), or (2) at a pH of less than 3.5.
6. The method according to any one of the preceding embodiments, wherein in step (b)(i) said pH is at least 3.5.
7. The method according to any one of the preceding embodiments, wherein in step (b)(i) the concentration of said acetonitrile is in the range of 30-100%(w/w) or at least 40%(w/w).
8. The method according to any one of the preceding embodiments, wherein in step (b)(ii) the concentration of said acetonitrile is at least 18%(w/w) or at least 20%(w/w).
9. The method according to any one of the preceding embodiments, wherein in step (b)(ii) the concentration of said acetonitrile is at least 30%(w/w), such as at least 33%(w/w) or at least 35%(w/w).
10. The method according to any one of the preceding embodiments, wherein in step (b)(ii) the concentration of said acetonitrile is at least 44%(w/w), such as at least 45%(w/w) or at least 46%(w/w).
11. The method according to any one of the preceding embodiments, wherein in step (b)(ii) the concentration of said acetonitrile is at least 55%(w/w).
12. The method according to any one of the preceding embodiments, wherein in step (b)(ii) the concentration of said acetonitrile is up to 98%(w/w), such as up to 95%(w/w) or up to 90%(w/w).
13. The method according to any one of the preceding embodiments, wherein in step (b)(ii) the concentration of said acetonitrile is up to 80%(w/w), such as up to 70%(w/w) or up to 60%(w/w).
14. The method according to any one of the preceding embodiments, wherein in step (b)(ii) the concentration of said acetonitrile is up to 60%(w/w), such as up to 40%(w/w) or up to 22%(w/w).
15. The method according to any one of the preceding embodiments, wherein in step (b)(ii) the concentration of said acetonitrile is 30-100%(w/w) or at least 40%(w/w).
16. The method according to any one of the preceding embodiments, wherein in step (b)(ii) said pH is less than 3.4, less than 3.3 or less than 3.2.
17. The method according to any one of the preceding embodiments, wherein in step (b)(ii) said pH is at least 1.0, at least 1.5, or at least 2.0.
18. The method according to any one of the preceding embodiments, wherein in step (b)(ii) said pH is at least 2.5, or at least 3.0.
19. The method according to any one of the preceding embodiments, wherein the temperature of said incubation in step (b)(i) is selected from the group consisting of 2-40° C., 40-85° C. or 10-50° C.
20. The method according to any one of the preceding embodiments, wherein in step (b)(i) said temperature is in the range of 5-50° C. or in the range of 10-40° C.
21. The method according to any one of the preceding embodiments, wherein in step (b)(i) said temperature is in the range of in the range of 15-30° C. or in the range of 21-25° C.
22. The method according to any one of the preceding embodiments, wherein in step (b)(i) said period is in the range of 5 min to 2 hours, such as in the range of 5-30 min, or in the range of 2-6 hours.
23. The method according to any one of the preceding embodiments, wherein in step (b)(i) said period is in the range of 1-48 hours or in the range of 8-40 hours.
24. The method according to any one of the preceding embodiments, wherein in step (b)(i) said period is in the range of 12-36 hours or in the range of 18-32 hours.
25. The method according to any one of the preceding embodiments, wherein in step (b)(iii)(2) the concentration of said acetonitrile is 0-20%(w/w) or at least 28%(w/w), such as 30-100%(w/w).
26. The method according to any one of the preceding embodiments, wherein in step (b)(iii)(2) the concentration of said acetonitrile is at least 40%(w/w) or at least 42%(w/w).
27. The method according to any one of the preceding embodiments, wherein in step (b)(iii)(2) the concentration of said acetonitrile is at least 45%(w/w) or at least 50%(w/w).
28. The method according to any one of the preceding embodiments, wherein in step (b)(iii)(2) the concentration of said acetonitrile is 1-18%(w/w) or 2-16%(w/w).
29. The method according to any one of the preceding embodiments, wherein in step (b)(ii) said temperature is in the range of 2-40° C., such as at least 5-30° C.
30. The method according to any one of the preceding embodiments, wherein in step (b)(ii) said concentration of acetonitrile is in the range of 15-100%(w/w).
31. The method according to any one of the preceding embodiments, wherein in step (b)(ii) said concentration of acetonitrile is at least 15%(w/w), such as at least 18%(w/w) or at least 20%(w/w).
32. The method according to any one of the preceding embodiments, wherein in step (b)(ii) said concentration of acetonitrile is no more than 99%(w/w), such as no more than 98% (w/w) or no more than 95%(w/w). 33. The method according to any one of the preceding embodiments, wherein in step (b)(ii) said concentration of acetonitrile is no more than 55%(w/w), such as no more than 40% (w/w) or no more than 22%(w/w).
34. The method according to any one of the preceding embodiments, wherein in step (b)(ii) said pH is in the range of 1-3.5.
35. The method according to any one of the preceding embodiments, wherein in step (b)(ii) or in step (b)(iii)(2) said pH is less than 3.4 or less than 3.3.
36. The method according to any one of the preceding embodiments, wherein in step (b)(ii) or in step (b)(iii)(2) said pH is less than 3.2, such as less than 3.1 or less than 3.0.
37. The method according to any one of the preceding embodiments, wherein in step (b)(i) or step (b)(iii), such as step (b)(iii)(1) or step (b)(iii)(2), said temperature is at least 40° C., such as at least 50° C. or at least 60° C.
38. The method according to any one of the preceding embodiments, wherein said pH and concentration of said organic solvent is as defined by Equation 32, 33, 34 as defined herein.
39. The method according to any one of the preceding embodiments, wherein said pH and concentration of said organic solvent is as defined by Equation 35, 36, 37 as defined herein.
40. The method according to any one of the preceding embodiments, wherein said pH and concentration of said organic solvent is as defined by Equation 65, 66, 67 as defined herein.
41. The method according to any one of the preceding embodiments, wherein said pH and concentration of said organic solvent is as defined by Equation 68, 69, or 70 as defined herein.
42. The method according to any one of the preceding embodiments, wherein said pH and concentration of said organic solvent is as defined by Equation 71, 72, or 73 as defined herein.
43. The method according to any one of the preceding embodiments, wherein said pH and concentration of said organic solvent is as defined by Equation 74, 75, or 76 as defined herein.
44. The method according to any one of the preceding embodiments, wherein said pH and concentration of said organic solvent is as defined by Equation 77, 78, or 79 as defined herein.
45. The method according to any one of the preceding embodiments, wherein said pH and concentration of said organic solvent is as defined by Equation 80, 81, 82, or 83 as defined herein.
46. The method according to any one of the preceding embodiments, wherein the organic solvent is DMF; and said incubating step (b) is for a period of at least 20 minutes and
   (i) at a pH of less than 5.0 and at a concentration of said DMF in the range of 5-28%(w/w); or
   (ii) at a pH in the range of 5.0-5.5 and at a concentration of said DMF in the range of 0-35%(w/w).
47. A method comprising the steps of:
(a) obtaining a first composition comprising undissolved and/or insoluble liraglutide and the organic solvent DMF, wherein said first composition is in the form of a solution or a suspension; and
(b) incubating said first composition for a period of at least 20 minutes and at a temperature in the range of 2-85° C.; and the incubation is carried out
   (i) at a pH of less than 5.0 and at a concentration of said DMF in the range of 5-28%(w/w); or
   (ii) at a pH in the range of 5.0-5.5 and at a concentration of said DMF in the range of 0-35%(w/w).
48. The method according to any one of the preceding embodiments, wherein in step (b)(i) said pH is less than 4 and optionally at least 1.
49. The method according to any one of the preceding embodiments, wherein in step (b)(i) said pH is less than 3.8, such as less than 3.7 or less than 3.6.
50. The method according to any one of the preceding embodiments, wherein in step (b)(i) the concentration of said DMF is in the range of 7-23%(w/w) or in the range of 8-26%(w/w).
51. The method according to any one of the preceding embodiments, wherein in step (b)(i) the concentration of said DMF is in the range of 9-25%(w/w) or in the range of 10-23%(w/w).
52. The method according to any one of the preceding embodiments, wherein in step (b)(i) said temperature is in the range of 5-50° C. or in the range of 10-40° C.
53. The method according to any one of the preceding embodiments, wherein in step (b)(i) said temperature is in the range of in the range of 15-30° C. or in the range of 21-25° C.
54. The method according to any one of the preceding embodiments, wherein in step (b)(i) said period is in the range of 1-48 hours or in the range of 8-40 hours.
55. The method according to any one of the preceding embodiments, wherein in step (b)(i) said period is in the range of 12-36 hours or in the range of 18-32 hours.
56. The method according to any one of the preceding embodiments, wherein said organic solvent is DMF, and in step (b)(i) the concentration of said DMF is in the range of 7-23%(w/w).
57. The method according to the preceding embodiment, wherein said period is at least 1 hour.
58. The method according to any one of the preceding embodiments, wherein said organic solvent is DMF, and in step (b)(i)
   a. said pH is less than 3.8,
   b. the concentration of said DMF is in the range of 9-25%(w/w), and
   c. said period is at least 8 hours.
59. The method according to any one of the preceding embodiments, wherein in step (b)(i) the concentration of said DMF is at least 8%(w/w) or at least 10%(w/w).
60. The method according to any one of the preceding embodiments, wherein in step (b)(i) the concentration of said DMF is up to 22%(w/w), up to 21%(w/w) or up to 20%(w/w).
61. The method according to any one of the preceding embodiments, wherein in step (b)(i) the pH is in the range of from 1.0 to less than 5.0.
62. The method according to any one of the preceding embodiments, wherein in step (b)(ii) the pH is at least 2.0, at least 2.5 or at least 3.0.
63. The method according to any one of the preceding embodiments, wherein in step (b)(ii) the pH is less than 4.8 or less than 4.9.
64. The method according to any one of the preceding embodiments, wherein in step (b)(ii) the pH is in the range of 4.8-6.0.
65. The method according to any one of the preceding embodiments, wherein in step (b)(ii) the pH is at least 4.8 or at least 4.9.
66. The method according to any one of the preceding embodiments, wherein in step (b)(ii) the pH is up to 5.8, up to 5.7, or up to 5.6.
67. The method according to any one of the preceding embodiments, wherein in step (b)(ii) the pH is up to 6.0 or up to 5.9.
68. The method according to any one of the preceding embodiments, wherein in step (b)(ii) the concentration of said DMF is at least 1%(w/w), at least 2%(w/w), or at least 3%(w/w).
69. The method according to any one of the preceding embodiments, wherein in step (b)(ii) the concentration of said DMF is at least 1%(w/w), at least 4%(w/w), or at least 5%(w/w).
70. The method according to any one of the preceding embodiments, wherein in step (b)(ii) the concentration of said DMF is up to 37%(w/w), up to 32%(w/w) or up to 30%(w/w).
71. The method according to any one of the preceding embodiments, wherein said pH and concentration of said organic solvent is as defined by Equation 84, 85, 86, or 87 as defined herein.
72. The method according to any one of the preceding embodiments, wherein in step (b) the incubation time is less than 8 hours.
73. The method according to any one of the preceding embodiments, wherein in step (b) the incubation time is less than 6 hours.
74. The method according to any one of the preceding embodiments, wherein in step (b) the incubation time is less than 5 hours.
75. The method according to any one of the preceding embodiments, wherein in step (b) the incubation time is in the range from 30 min to less than 8 hours. 76. The method according to any one of the preceding embodiments, wherein in step (b) the incubation time is at least 1 hour.
77. The method according to any one of the preceding embodiments, wherein in step (b) the incubation time is at least 1.5 hours.
78. The method according to any one of the preceding embodiments, wherein in step (b) if the incubation time is at least 8 hours, then the incubation is carried out
   (i) at a pH of less than 5.0 and at a concentration of said DMF in the range of 2-40%(w/w); or
   (ii) at a pH in the range of 5.0-6.0 and at a concentration of said DMF in the range of 0-40%(w/w).
79. The method according to any one of the preceding embodiments, wherein in step (b)(i) said DMF is at a concentration at least 3%(w/w), at least 4%(w/w), or at least 5%(w/w).
80. The method according to any one of the preceding embodiments, wherein in step (b)(i) said DMF is at a concentration up to 35%(w/w), up to 30%(w/w), or up to 28%(w/w).
81. The method according to any one of the preceding embodiments, wherein in step (b)(i) said DMF is at a concentration up to 27%(w/w), up to 26%(w/w), or up to 25%(w/w).
82. The method according to any one of the preceding embodiments, wherein in step (b)(i) said pH is less than 4.5, less than 4.4 or less than 4.3.
83. The method according to any one of the preceding embodiments, wherein in step (b)(i) said pH is less than 4.2, less than 4.1 or less than 4.0.
84. The method according to any one of the preceding embodiments, wherein in step (b)(i) said pH is less than 3.9, less than 3.8 or less than 3.7.
85. The method according to any one of the preceding embodiments, wherein in step (b)(i) said pH is at least 1.0, at least 1.5, or at least 2.0.
86. The method according to any one of the preceding embodiments, wherein in step (b)(i) said pH is at least 2.5, or at least 3.0.
87. The method according to any one of the preceding embodiments, wherein in step (b)(ii) if pH is less than 5.5 then the concentration of said DMF is in the range of 0-20%(w/w).
88. The method according to any one of the preceding embodiments, wherein in step (b)(ii) if pH is less than 5.4 or less than 5.3 then the concentration of said DMF is in the range of 0-20%(w/w).
89. The method according to any one of the preceding embodiments, wherein in step (b)(ii) the concentration of said DMF is up to 20%(w/w) or up to 19%(w/w).
90. The method according to any one of the preceding embodiments, wherein in step (b)(ii) the concentration of said DMF is up to 18%(w/w) or up to 17%(w/w).
91. The method according to any one of the preceding embodiments, wherein in step (b) the incubation time is at least 12 hours, at least 16 hours, or at least 20 hours.
92. The method according to any one of the preceding embodiments, wherein in step (b) the incubation time is in the range from 8 to 48 hours.
93. The method according to any one of the preceding embodiments, wherein in step (b) the incubation time is less than 36 hours or less than 28 hours.
94. The method according to any one of the preceding embodiments, wherein in step (b) said pH is less than 5.0.
95. The method according to any one of the preceding embodiments, wherein in step (b) said pH is less than 4.5 or less than 4.0.
96. The method according to any one of the preceding embodiments, wherein in step (b) said pH is less than 3.5 or less than 3.0.
97. The method according to any one of the preceding embodiments, wherein in step (b) said pH is at least 1.0.
98. The method according to any one of the preceding embodiments, wherein said temperature is 10-50° C.
99. The method according to any one of the preceding embodiments, wherein said temperature is 15-40° C.
100. The method according to any one of the preceding embodiments, wherein the temperature is 20-30° C. or 21-25° C.
101. The method according to any one of the preceding embodiments, wherein said temperature is 10-50° C.
102. A method comprising the steps of:
(a) obtaining a first composition comprising undissolved and/or insoluble liraglutide and DMF, wherein said first composition is in the form of a solution or a suspension; and
(b) incubating said solution for a period of at least 5 minutes at a temperature of less than 50° C.; and (i) if the incubation temperature is at least 15° C., then the incubation is carried out (1) above pH 6.5, or (2) if the incubation is carried out for more than 4 hours then wherein the concentration of said organic solvent in relation to the pH of the solution (pH) is in the range of c_{(organic solvent)} to 80%(w/w), wherein c_{(organic solvent)}≥−13.3*pH+123; or (ii) if the incubation temperature is less than 15° C., then the incubation is carried out above pH 7.0, provided that if the incubation time is less than 30 minutes then the concentration of said organic solvent is less than 25%(w/w); with the proviso that if the organic solvent is phenol then the concentration of said organic solvent is at least 1%(w/w); wherein said method increases the amount of dissolved liraglutide.
103. The method according to any one of the preceding embodiments, wherein in step (b)(i)(2) the concentration of said organic solvent in relation to the pH of the solution (pH) is in the range of c(organic solvent) to 80%(w/w), wherein c(organic solvent)≥−40*pH +310.
104. The method according to any one of the preceding embodiments, wherein in step (i)(2) the concentration of said organic solvent in relation to the pH of the solution (pH) is in the range of c(organic solvent) to 80%(w/w), wherein c(organic solvent)≥−40*pH+310.
105. The method according to any one of the preceding embodiments, wherein in step (ii) the concentration of said organic solvent is less than 23%(w/w), such as less than 22%(w/w) or less than 21%(w/w).
106. The method according to any one of the preceding embodiments, wherein in step (ii) the concentration of said organic solvent is less than 20%(w/w), such as less than 19% (w/w) or less than 18%(w/w).
107. The method according to any one of the preceding embodiments, wherein said pH of step (b) is in the range of 6.5-12.5 or 7.0-12.5
108. The method according to any one of the preceding embodiments, wherein said pH of step (b) is at least 6.7, such as at least 6.8, at least 6.9, or at least 7.0.
109. The method according to any one of the preceding embodiments, wherein said temperature of step (b) is in the range of 2° C.-50° C., such as 4° C.-40° C., or 5° C.-30° C.
110. The method according to embodiment 7, wherein said temperature of step (b) is less than 40° C., such as less than 40° C., less than 30° C., or less than 25° C.
111. The method according to any one of the preceding embodiments, wherein said temperature of step (b) is at least 4° C., such as at least 10° C., at least 15° C., or at least 20° C.
112. The method according to any one of the preceding claims, wherein said method comprises the following additional step: (c) reducing the concentration of said organic solvent in the first composition subjected to step (b), and optionally isolating liraglutide.
113. The method according to any one of the preceding claims, wherein said method provides an increase in dissolved GLP-1 peptide, such as liraglutide, by at least 0.5 mg/ml after an incubation period of 3 hours, optionally as described in Assay (I) herein.
114. The method according to any one of the preceding claims, wherein said first composition of step (a) is prepared from liraglutide (i) in the form of a precipitated powder or (ii) is undissolved and/or insoluble in suspension or solution.
115. The method according to any one of the preceding claims, wherein the pH of the first composition of step (a) is in the range of pH 1-13.
116. The method according to any one of the preceding claims, wherein the temperature of said incubation in step (b) is
(A) a temperature between the freezing point and the boiling point of said first composition comprising said one or more organic solvents,
(B) above the freezing point of the solution subjected to said incubation and below 50° C., or
(C) in the range of 50-85° C.
117. The method according to any one of the preceding claims, wherein the organic solvent is selected from the group consisting of ethanol, 2-propanol, acetonitrile, phenol, and DMSO.
118. The method according to any one of the preceding claims, wherein the first composition of step (a) has a concentration of said organic solvent is in the range of 0.01 to 80%(w/w).
119. The method according to any one of the preceding embodiments, wherein said first composition is in the form of a solution or a suspension.
120. The method according to any one of the preceding embodiments, wherein said prior to step (a) said undissolved and/or insoluble liraglutide is present in an initial composition, and said initial composition comprises at least 0.1%(w/w) undissolved and/or insoluble liraglutide.
121. The method according to any one of the preceding embodiments, wherein said prior to step (a) said undissolved and/or insoluble liraglutide is present in an initial composition, and said initial composition comprises at least at least 1%(w/w) or at least 5%(w/w) undissolved and/or insoluble liraglutide.
122. The method according to any one of the preceding embodiments, wherein said prior to step (a) said undissolved and/or insoluble liraglutide is present in an initial composition, and said initial composition comprises at least at least 10%(w/w) or at least 50%(w/w) undissolved and/or insoluble liraglutide.
123. The method according to any one of the preceding embodiments, wherein said incubation is as defined in the equations of Tables 1 and 2 herein.
124. The method according to any one of the preceding embodiments, wherein said method subsequent to step (b) comprises a further step (c), said step (c) comprising , in any sequence, one or more selected from the group consisting of:
   (1) reducing the concentration of said organic solvent,
   (2) neutralising pH,
   (3) reducing the temperature,
   (4) reducing the concentration of said organic solvent, and
   (5) optionally isolating liraglutide.
125. The method according to any one of the preceding embodiments, wherein after step (b) said method comprises one or more of the following additional steps, in any sequence, using the composition obtained from step (b):
   (c1) reducing the concentration of said organic solvent in said composition from step (b),
   (c2) neutralising pH of said composition from step (b),
   (c3) reducing the temperature of said composition from step (b), and
   (c4) isolating liraglutide.
126. The method according to any one of the preceding embodiments, wherein after step
(b) said method comprises the following additional step:
(c) reducing the concentration of said organic solvent in the composition obtained from step
(b), and optionally isolating liraglutide.
127. The method according to any one of the preceding embodiments, wherein said neutralising is to a pH in the range of 7.0-10.0.
128. The method according to any one of the preceding embodiments, wherein said neutralising is to a pH in the range of 7.4-9.0 or 7.8-8.4.
129. The method according to any one of the preceding embodiments, wherein said reducing the temperature is to a temperature in the range of 2-40° C., such as 4-35° C. or 5-30° C.
130. The method according to any one of the preceding embodiments, wherein the composition obtained from step (b) is referred to as the second composition.
131. The method according to any one of the preceding embodiments, wherein said method increases the amount of dissolved GLP-1 peptide, such as liraglutide.
132. The method according to any one of the preceding embodiments, wherein said method provides an increase in dissolved GLP-1 peptide by at least 10%(w/w), such as at least 20%(w/w) or at least 30%(w/w).
133. The method according to any one of the preceding embodiments, wherein said method provides an increase in dissolved GLP-1 peptide, such as liraglutide, by at least 0.2 mg/ml after an incubation period of 3 hours, optionally determined as described in Assay (I) herein.
134. The method according to any one of the preceding embodiments, wherein said method provides an increase in dissolved GLP-1 peptide, such as liraglutide, by 0.2-1.0 mg/ml after an incubation period of 3 hours, optionally determined as described in Assay (I) herein.
135. The method according to any one of the preceding embodiments, wherein said method provides an increase in dissolved GLP-1 peptide, such as liraglutide, by at least 0.3 mg/ml after an incubation period of 3 hours, optionally determined as described in Assay (I) herein.
136. The method according to any one of the preceding embodiments, wherein said method provides an increase in dissolved GLP-1 peptide, such as liraglutide, by at least 0.4 mg/ml after an incubation period of 3 hours, optionally determined as described in Assay (I) herein.
137. The method according to any one of the preceding embodiments, wherein said method provides an increase in dissolved GLP-1 peptide, such as liraglutide, by at least 0.5 mg/ml after an incubation period of 3 hours, optionally determined as described in Assay (I) herein.
138. The method according to any one of the preceding embodiments, wherein said method provides an increase in dissolved GLP-1 peptide, such as liraglutide, by at least 0.6 mg/ml after an incubation period of 3 hours, optionally determined as described in Assay (I) herein.
139. The method according to any one of the preceding embodiments, wherein said method provides an increase in dissolved GLP-1 peptide, such as liraglutide, by at least 0.7 mg/ml after an incubation period of 3 hours, optionally determined as described in Assay (I) herein.
140. The method according to any one of the preceding embodiments, wherein said method provides an increase in dissolved GLP-1 peptide, such as liraglutide, by at least 0.8 mg/ml after an incubation period of 3 hours, optionally determined as described in Assay (I) herein.
141. The method according to any one of the preceding embodiments, wherein said method provides an increase in dissolved GLP-1 peptide, such as liraglutide, by at least 0.5 mg/ml after an incubation period of 3 hours, optionally as described in Assay (I) herein.
142. The method according to any one of the preceding embodiments, wherein the concentration of dissolved GLP-1 peptide is determined as described in Assay (I) herein, and optionally after an incubation period of 3 hours.
143. The method according to any one of the preceding embodiments, wherein said GLP-1 peptide is liraglutide.
144. The method according to any one of the preceding embodiments, wherein said composition of step (a) is prepared from liraglutide in the form of (i) a precipitate, (ii) a powder or (iii) is undissolved and/or insoluble in suspension or solution.
145. The method according to any one of the preceding embodiments, wherein said composition of step (a) is prepared from an initial composition comprising undissolved and/or insoluble liraglutide in suspension or solution.
146. The method according to any one of the preceding embodiments, wherein said composition of step (a) is prepared from an initial composition comprising undissolved liraglutide in suspension or solution.
147. The method according to any one of the preceding embodiments, wherein said composition of step (a) is prepared from an initial composition comprising insoluble liraglutide in suspension or solution.
148. The method according to any one of the preceding embodiments, wherein said first composition is in the form of a suspension.
149. The method according to any one of the preceding embodiments, wherein said first composition is in the form of a solution.
150. The method according to any one of the preceding embodiments, wherein the pH of the composition subjected to said incubation in step (b) is in the range of pH 1-13.
151. The method according to any one of the preceding embodiments, wherein the pH of the solution subjected to said incubation in step (b) is in the range of pH 1-13.
152. The method according to any one of the preceding embodiments, wherein the pH of the composition subjected to said incubation in step (b) is in the range of pH 3-11.
153. The method according to any one of the preceding embodiments, wherein said incubation is carried out at a temperature between the freezing point and the boiling point of the solution comprising said one or more organic solvents.
154. The method according to any one of the preceding embodiments, wherein the temperature of said incubation in step (b) is above the freezing point of the solution subjected to said incubation and below 50° C.
155. The method according to any one of the preceding embodiments, wherein the temperature of said incubation in step (b) is selected from the group consisting of 2-40° C., 40-85° C. or 10-50° C.
156. The method according to any one of the preceding embodiments, wherein the temperature of said incubation in step (b) is in the range of 50-85° C.
157. The method according to any one of the preceding embodiments, wherein said increases storage stability of liraglutide in solution refers to at least 90%(w/w) of said liraglutide remains in solution following storage at 5° C. for 1 or 2 years.
158. The method according to any one of the preceding embodiments, wherein said increases storage stability of liraglutide in solution refers to at least 90%(w/w) of said liraglutide remains in solution following storage at room temperature for two months.
159. The method according to any one of the preceding embodiments, wherein said increases storage stability of liraglutide in solution refers to at least 90%(w/w) of said liraglutide remains in solution following storage at 5° C. for 24 hours or 1 week.
160. The method according to any one of the preceding embodiments, wherein at least 95%(w/w) of said liraglutide remains in solution following storage.
161. The method according to any one of the preceding embodiments, wherein at least 97%(w/w) of said liraglutide remains in solution following storage.
162. The method according to any one of the preceding embodiments, wherein at least 99%(w/w) of said liraglutide remains in solution following storage.
163. The method according to any one of the preceding claims, wherein said method comprises a step of obtaining a composition comprising substantially no acetonitrile or DMF.
164. A stable pharmaceutical composition comprising a GLP-1 peptide obtained by the method as defined in any one of the preceding embodiments and said pharmaceutical composition further comprises one or more pharmaceutically acceptable excipients.

### EXAMPLES

### List of Abbreviations

CAPS: N-cyclohexyl-3-am inopropanesulfonic acid

DMSO: Dimethyl sulfoxide

EtOH: Ethanol

HEPES: 2-[4-(2-hydroxyethyl)piperazin-1-yl]ethanesulfonic acid

MeCN: Acetonitrile

MES: 2-(N-morpholino)ethanesulfonic acid

ISOP: Isopropanol

### General Methods

Assay (I): Determination of Solubility

### 96-Well Plate Preparation

96-well plates containing target peptide in a solid form were prepared in one of two ways:

Method 1 (evaporation): Addition of 100 μl stock solution of said peptide at a concentration of 4-6 mg/ml to each well of a 96-well half area plate (150 μl well size). Subsequently the solvent was evaporated by applying a vacuum until the solvent was completely removed.

Method 2 (precipitation): Addition of a certain volume of a stock solution of said peptide containing to each well of a 96-well half area plate followed by the addition of a certain volume of concentrated phosphoric acid to obtain a pH of 4.6 followed by addition of a certain volume of ethanol resulting in a final ethanol concentration of 20% v/v. Subsequently the plate was stored at 4° C. for 1 h and then centrifuged at 4,000 rpm for 20 min. Finally the supernatant was removed. The concentration of the stock solution was chosen in a way that the final amount of peptide after supernatant removal was >0.4 mg per well.

In both cases the stock solution was prepared by adding de-ionized water to a certain amount of powder of said peptide followed by a stepwise addition of small amounts of a concentrated sodium hydroxide solution until said peptide was completely in solution.

### Solubility Measurement

To determine solubility of a target peptide at different pH and organic solvent concentrations, two different buffer systems were used in independent experiments to cover the whole range from pH 3.0 to pH 11.0. Buffer system 1 covers pH 3.0-7.5 and buffer system 2 covers pH 7.5-11.0. For each buffer system 96 different solvent compositions were premixed in the following way:
1. A low pH buffer (adjusted to either pH 3.0 or pH 7.5) and a high pH buffer (adjusted to either pH 7.5 or pH 11.0) were mixed at 8 different ratios to obtain values between pH 3.0 and pH 7.5 or pH 7.5 and pH 11.0, respectively. The total volume after mixing was 300 μl.
2. 12 different amounts of organic solvent were then added to these buffers to obtain organic solvent (e.g. ethanol) concentrations between 0 and 70% w/w.
3. Water was then added to obtain a total weight of 1.0 g of solvent in each well.

After preparing the 96 different solvent systems, 100 μl of each system was transferred to the 96-well plate containing the solid form of the target peptide (prepared as described in the section “96-well plate preparation”). The plate was then stored at a given temperature (between 5° C. and 70° C.) for a given time on a rotating shaker set to 400 rpm. At regular time intervals the plate was centrifuged (4,000 rpm for 10 min) and a 10 μl sample was taken from each well. The samples were then analysed by UV absorption at 280 nm for peptide quantification. Peptide concentration results are given as UV absorption at 280 nm of the sample subtracted UV absorption at 280 nm of a well comprising 10 μl water.

The buffers used were as follows:
Buffer system 1 (for the pH range from pH 3.0-7.5)

| Component | Concentration [mM] |
|---|---|
| Formic acid | 91 |
| Acetic acid | 60 |
| MES | 88 |
| HEPES | 94 |

Buffer system 2 (for the pH ranae from pH 7.5-11.0)

| Component | Concentration [mM] |
|---|---|
| HEPES | 121 |
| Histidine | 85 |
| CAPS | 128 |

### Assay (II): In Vitro Potency of GLP-1 Peptides (CRE Luciferase; Whole Cells)

The purpose of this example is to test the activity, also referred to as potency, of GLP-1 peptides in vitro. The in vitro potency is the measure of human GLP-1 receptor activation in a whole cell assay.

Principle: In vitro potency is determined by measuring the response of the human GLP-1 receptor in a reporter gene assay. The assay is performed in a stably transfected BHK cell line that expresses the human GLP-1 receptor and contains the DNA for the cAMP response element (CRE) coupled to a promoter and the gene for firefly luciferase (CRE luciferase). When the human GLP-1 receptor is activated it results in the production of cAMP, which in turn results in the luciferase protein being expressed. When assay incubation is completed the luciferase substrate (luciferin) is added and the enzyme converts luciferin to oxyluciferin to produce bioluminescence. The luminescence is measured as the readout for the assay.

Cell culture and preparation: The cells used in this assay (e.g. clone FCW467-12A/KZ10-1) are BHK cells with BHKTS13 as a parent cell line. The cells are derived from a clone (e.g. FCW467-12A) that expresses the human GLP-1 receptor and are established by further transfection with CRE luciferase to obtain the current clone. The cells are cultured at 5% CO₂ in Cell Culture Medium. They are aliquoted and stored in liquid nitrogen. Before each assay an aliquot is taken up and washed twice in PBS before being suspended at the desired concentration in the assay specific buffer. For 96-well plates the suspension is made to give a final concentration of 5×10³ cells/well.

Materials: The following chemicals may be used in the assay: Pluronic F-68 (10%) (Gibco 2404), ovalbumin (Sigma A5503), DMEM w/o phenol red (Gibco 11880-028), 1 M Hepes (Gibco 15630), Glutamax 100× (Gibco 35050) and steadylite plus (PerkinElmer 6016757).

Buffers: Cell Culture Medium consists of DMEM medium with 10% FBS (Fetal Bovine Serum; Invitrogen 16140-071), 1 mg/ml G418 (Invitrogen 15140-122), 240 nM MTX (methotrexate; Sigma M9929) and 1% pen/strep (penicillin/streptomycin; Invitrogen 15140-122). Assay Medium consists of DMEM w/o phenol red, 10 mM Hepes and lx Glutamax. Assay Buffer consists of 2% ovalbumin and 0.2% Pluronic F-68 in Assay Medium.

Procedure: 1) Cell stocks are thawed in a 37° C. water bath. 2) Cells are washed three times in PBS. 3) The cells are counted and adjusted to 5×10³ cells/50 μl (1×10⁵ cells/ml) in Assay Medium; a 50 μlaliquot of cells is transferred to each well in the assay plate. 4) Stocks of the test compounds (and reference compounds, if any) are diluted to a concentration of 0.2 μM in Assay Buffer; compounds are diluted 10-fold to give the following concentrations: 2×10⁻⁷ M, 2×10⁻⁵ M; 2×10⁻⁹ M, 2×10⁻¹⁰ M, 2×10⁻¹¹ M, 2×10⁻¹² M, 2×10⁻¹³ M, and 2×10⁻¹⁴ M. 5) A 50 μl aliquot of compound or blank is transferred from the dilution plate to the assay plate; compounds are tested at the following final concentrations: 1×10⁻⁷ M, 1×10⁻⁵ M; 1×10⁻⁹ M, 1×10⁻¹⁰ M, 1×10⁻¹¹ M, 1×10⁻¹² M, 1×10⁻¹³ M, and 1×10⁻¹⁴ M. 6) The assay plate is incubated for 3 h in a 5% CO₂ incubator at 37° C. 7) The assay plate is removed from the incubator and allowed to stand at room temperature for 15 min. 8) A 100 μl aliquot of steadylite plus reagent is added to each well of the assay plate (reagent was light sensitive). 9) Each assay plate is covered with aluminium foil to protect it from light and shaken for 30 min at room temperature. 10) Each assay plate is read in a plate reading instrument, e.g. Packard TopCount NXT.

Calculations and Results: The data from the plate reading instrument are transferred to software (e.g. GraphPad Prism) which performs a non-linear regression (log(agonist) vs response) and calculates EC50 values which may be reported in pM. A minimum of two replicates is measured for each sample. The reported values are averages of the replicates.

### Example 1

### Liraglutide Solubility

Solubility of liraglutide was determined using Assay (I) described herein. Liraglutide was prepared using Method 1 (evaporation) or Method 2 (precipitation) as described in subsection “96-well plate preparation”. The results as well as the specific parameters used are shown in Table 3a and Table 3b.

TABLE 3a
Treatment conditions using Method 2 (precipitation) and results in the
form of GLP-1 peptide concentration at specific incubation time.

| | | | | GLP-1 peptide concentration [mg/ml] | | | | | |
|---|---|---|---|---|---|---|---|---|---|
| Organic solvent | | | | | | | | | |
| | Conc. | | Temp. | Before removal of outliers | | | After removal of outliers | | |
| Type | [% w/w] | pH | [° C.] | 5 min | 180 min | 24 h | 5 min | 180 min | 24 h |
| MeCN | 0.0 | 3.00 | 5 | 0.08 | 0.11 | 0.14 | 0.1 | 0.1 | 0.1 |
| MeCN | 0.0 | 3.64 | 5 | 0.36 | 0.06 | 0.03 | 0.4 | 0.1 | 0.0 |
| MeCN | 0.0 | 4.27 | 5 | 0.53 | 0.06 | 0.04 | 0.5 | 0.1 | 0.0 |
| MeCN | 0.0 | 4.93 | 5 | 0.71 | 0.04 | 0.03 | 0.7 | 0.0 | 0.0 |
| MeCN | 0.0 | 5.57 | 5 | 0.49 | 0.04 | 0.01 | 0.5 | 0.0 | 0.0 |
| MeCN | 0.0 | 6.22 | 5 | 0.18 | 0.06 | 0.01 | 0.2 | 0.1 | 0.0 |
| MeCN | 0.0 | 6.86 | 5 | 1.39 | 1.64 | 1.53 | 1.4 | 1.6 | 1.5 |
| MeCN | 0.0 | 7.46 | 5 | 2.37 | >4.0 | >4.0 | 3.7 | >4.0 | >4.0 |
| MeCN | 6.4 | 3.00 | 5 | 0.08 | 0.11 | 0.08 | 0.1 | 0.1 | 0.1 |
| MeCN | 6.4 | 3.64 | 5 | 0.14 | 0.04 | 0.04 | 0.1 | 0.0 | 0.0 |
| MeCN | 6.4 | 4.27 | 5 | 0.22 | 0.07 | 0.06 | 0.2 | 0.1 | 0.1 |
| MeCN | 6.4 | 4.93 | 5 | 0.14 | 0.06 | 0.04 | 0.1 | 0.1 | 0.0 |
| MeCN | 6.4 | 5.57 | 5 | 0.12 | 0.04 | 0.03 | 0.1 | 0.0 | 0.0 |
| MeCN | 6.4 | 6.22 | 5 | 0.08 | 0.08 | 0.01 | 0.1 | 0.1 | 0.0 |
| MeCN | 6.4 | 6.86 | 5 | 1.40 | 2.30 | 2.30 | 1.4 | 2.3 | 2.3 |
| MeCN | 6.4 | 7.46 | 5 | 2.39 | >4.0 | 3.87 | 3.7 | >4.0 | >4.0 |
| MeCN | 12.7 | 3.00 | 5 | 0.08 | 0.12 | 0.12 | 0.1 | 0.1 | 0.1 |
| MeCN | 12.7 | 3.64 | 5 | 0.06 | 0.04 | 0.01 | 0.1 | 0.0 | 0.0 |
| MeCN | 12.7 | 4.27 | 5 | 0.07 | 0.04 | 0.04 | 0.1 | 0.0 | 0.0 |
| MeCN | 12.7 | 4.93 | 5 | 0.07 | 0.04 | 0.01 | 0.1 | 0.0 | 0.0 |
| MeCN | 12.7 | 5.57 | 5 | 0.08 | 0.06 | 0.03 | 0.1 | 0.1 | 0.0 |
| MeCN | 12.7 | 6.22 | 5 | 0.22 | 0.15 | 0.08 | 0.2 | 0.2 | 0.1 |
| MeCN | 12.7 | 6.86 | 5 | 1.93 | >4.0 | >4.0 | 1.9 | >4.0 | >4.0 |
| MeCN | 12.7 | 7.46 | 5 | 2.00 | >4.0 | >4.0 | 2.7 | >4.0 | 4.0 |
| MeCN | 19.1 | 3.00 | 5 | 0.85 | 0.74 | 0.67 | 0.8 | 0.7 | 0.7 |
| MeCN | 19.1 | 3.64 | 5 | 0.08 | 0.07 | 0.04 | 0.1 | 0.1 | 0.0 |
| MeCN | 19.1 | 4.27 | 5 | 0.06 | 0.06 | 0.04 | 0.1 | 0.1 | 0.0 |
| MeCN | 19.1 | 4.93 | 5 | 0.07 | 0.04 | 0.03 | 0.1 | 0.0 | 0.0 |
| MeCN | 19.1 | 5.57 | 5 | 0.07 | 0.06 | 0.06 | 0.1 | 0.1 | 0.1 |
| MeCN | 19.1 | 6.22 | 5 | 0.90 | 0.75 | 0.39 | 0.9 | 0.7 | 0.4 |
| MeCN | 19.1 | 6.86 | 5 | 1.53 | >4.0 | >4.0 | 1.5 | >4.0 | >4.0 |
| MeCN | 19.1 | 7.46 | 5 | 1.65 | >4.0 | >4.0 | 1.9 | >4.0 | 3.2 |
| MeCN | 25.5 | 3.00 | 5 | 1.71 | 3.69 | >4.0 | 1.7 | 3.7 | >4.0 |
| MeCN | 25.5 | 3.64 | 5 | 0.07 | 0.07 | 0.04 | 0.1 | 0.1 | 0.0 |
| MeCN | 25.5 | 4.27 | 5 | 0.06 | 0.06 | 0.04 | 0.1 | 0.1 | 0.0 |
| MeCN | 25.5 | 4.93 | 5 | 0.06 | 0.07 | 0.04 | 0.1 | 0.1 | 0.0 |
| MeCN | 25.5 | 5.57 | 5 | 0.11 | 0.07 | 0.08 | 0.1 | 0.1 | 0.1 |
| MeCN | 25.5 | 6.22 | 5 | 0.49 | 0.92 | 0.40 | 0.5 | 0.9 | 0.4 |
| MeCN | 25.5 | 6.86 | 5 | 1.58 | 2.69 | 2.43 | 1.6 | 2.7 | 2.4 |
| MeCN | 25.5 | 7.46 | 5 | 2.85 | >4.0 | >4.0 | 2.7 | >4.0 | 2.4 |
| MeCN | 31.8 | 3.00 | 5 | 1.74 | 3.93 | 3.61 | 1.7 | 3.9 | 3.6 |
| MeCN | 31.8 | 3.64 | 5 | 0.19 | 0.08 | 0.07 | 0.2 | 0.1 | 0.1 |
| MeCN | 31.8 | 4.27 | 5 | 0.07 | 0.08 | 0.10 | 0.1 | 0.1 | 0.1 |
| MeCN | 31.8 | 4.93 | 5 | 0.06 | 0.07 | 0.04 | 0.1 | 0.1 | 0.0 |
| MeCN | 31.8 | 5.57 | 5 | 0.25 | 0.18 | 0.10 | 0.2 | 0.2 | 0.1 |
| MeCN | 31.8 | 6.22 | 5 | 0.46 | 0.53 | 0.39 | 0.5 | 0.5 | 0.4 |
| MeCN | 31.8 | 6.86 | 5 | 0.89 | 1.43 | 1.19 | 0.9 | 1.4 | 1.2 |
| MeCN | 31.8 | 7.46 | 5 | 2.12 | 3.94 | 3.47 | 2.7 | 3.1 | 2.2 |
| MeCN | 38.2 | 3.00 | 5 | 1.24 | 2.72 | 2.62 | 1.2 | 2.7 | 2.6 |
| MeCN | 38.2 | 3.64 | 5 | 0.29 | 0.26 | 0.11 | 0.3 | 0.3 | 0.1 |
| MeCN | 38.2 | 4.27 | 5 | 0.21 | 0.15 | 0.12 | 0.2 | 0.2 | 0.1 |
| MeCN | 38.2 | 4.93 | 5 | 0.18 | 0.12 | 0.07 | 0.2 | 0.1 | 0.1 |
| MeCN | 38.2 | 5.57 | 5 | 0.25 | 0.31 | 0.24 | 0.2 | 0.3 | 0.2 |
| MeCN | 38.2 | 6.22 | 5 | 0.35 | 0.42 | 0.37 | 0.3 | 0.4 | 0.4 |
| MeCN | 38.2 | 6.86 | 5 | 0.83 | 1.12 | 1.15 | 0.8 | 1.1 | 1.2 |
| MeCN | 38.2 | 7.46 | 5 | 1.50 | 2.86 | 2.37 | 2.3 | 3.8 | 3.1 |
| MeCN | 44.5 | 3.00 | 5 | 1.06 | 1.35 | 1.08 | 1.1 | 1.3 | 1.1 |
| MeCN | 44.5 | 3.64 | 5 | 0.31 | 0.37 | 0.17 | 0.3 | 0.4 | 0.2 |
| MeCN | 44.5 | 4.27 | 5 | 0.22 | 0.32 | 0.11 | 0.2 | 0.3 | 0.1 |
| MeCN | 44.5 | 4.93 | 5 | 0.25 | 0.21 | 0.17 | 0.2 | 0.2 | 0.2 |
| MeCN | 44.5 | 5.57 | 5 | 0.24 | 0.28 | 0.29 | 0.2 | 0.3 | 0.3 |
| MeCN | 44.5 | 6.22 | 5 | 0.42 | 0.50 | 0.54 | 0.4 | 0.5 | 0.5 |
| MeCN | 44.5 | 6.86 | 5 | 0.58 | 1.00 | 1.03 | 0.6 | 1.0 | 1.0 |
| MeCN | 44.5 | 7.46 | 5 | 0.68 | 1.99 | 1.19 | 1.5 | 2.7 | 3.2 |
| MeCN | 50.9 | 3.00 | 5 | 0.94 | 0.85 | 0.62 | 0.9 | 0.8 | 0.6 |
| MeCN | 50.9 | 3.64 | 5 | 0.28 | 0.47 | 0.37 | 0.3 | 0.5 | 0.4 |
| MeCN | 50.9 | 4.27 | 5 | 0.18 | 0.28 | 0.22 | 0.2 | 0.3 | 0.2 |
| MeCN | 50.9 | 4.93 | 5 | 0.26 | 0.33 | 0.28 | 0.3 | 0.3 | 0.3 |
| MeCN | 50.9 | 5.57 | 5 | 0.26 | 0.40 | 0.36 | 0.3 | 0.4 | 0.4 |
| MeCN | 50.9 | 6.22 | 5 | 0.32 | 0.47 | 0.44 | 0.3 | 0.5 | 0.4 |
| MeCN | 50.9 | 6.86 | 5 | 0.36 | 0.65 | 0.81 | 0.4 | 0.7 | 0.8 |
| MeCN | 50.9 | 7.46 | 5 | 0.44 | 0.94 | 1.26 | 2.2 | 1.8 | 2.5 |
| MeCN | 57.3 | 3.00 | 5 | 1.32 | 1.07 | 0.33 | 1.3 | 1.1 | 0.3 |
| MeCN | 57.3 | 3.64 | 5 | 0.36 | 0.47 | 0.51 | 0.4 | 0.5 | 0.5 |
| MeCN | 57.3 | 4.27 | 5 | 0.26 | 0.39 | 0.47 | 0.3 | 0.4 | 0.5 |
| MeCN | 57.3 | 4.93 | 5 | 0.19 | 0.31 | 0.36 | 0.2 | 0.3 | 0.4 |
| MeCN | 57.3 | 5.57 | 5 | 0.17 | 0.29 | 0.40 | 0.2 | 0.3 | 0.4 |
| MeCN | 57.3 | 6.22 | 5 | 0.22 | 0.40 | 0.46 | 0.2 | 0.4 | 0.5 |
| MeCN | 57.3 | 6.86 | 5 | 0.32 | 0.57 | 0.74 | 0.3 | 0.6 | 0.7 |
| MeCN | 57.3 | 7.46 | 5 | 0.29 | 0.69 | 1.19 | 1.1 | 2.2 | 3.2 |
| MeCN | 63.6 | 3.00 | 5 | 1.22 | 1.29 | 0.40 | 1.2 | 1.3 | 0.4 |
| MeCN | 63.6 | 3.64 | 5 | 0.32 | 0.54 | 0.67 | 0.3 | 0.5 | 0.7 |
| MeCN | 63.6 | 4.27 | 5 | 0.24 | 0.37 | 0.39 | 0.2 | 0.4 | 0.4 |
| MeCN | 63.6 | 4.93 | 5 | 0.21 | 0.29 | 0.42 | 0.2 | 0.3 | 0.4 |
| MeCN | 63.6 | 5.57 | 5 | 0.25 | 0.37 | 0.51 | 0.2 | 0.4 | 0.5 |
| MeCN | 63.6 | 6.22 | 5 | 0.19 | 0.33 | 0.44 | 0.2 | 0.3 | 0.4 |
| MeCN | 63.6 | 6.86 | 5 | 0.29 | 0.54 | 0.72 | 0.3 | 0.5 | 0.7 |
| MeCN | 63.6 | 7.46 | 5 | 0.25 | 0.60 | 0.85 | 0.9 | 1.0 | 2.4 |
| MeCN | 70.0 | 3.00 | 5 | 1.32 | 1.87 | 1.03 | 1.3 | 1.9 | 1.0 |
| MeCN | 70.0 | 3.64 | 5 | 0.32 | 0.54 | 0.68 | 0.3 | 0.5 | 0.7 |
| MeCN | 70.0 | 4.27 | 5 | 0.22 | 0.39 | 0.46 | 0.2 | 0.4 | 0.5 |
| MeCN | 70.0 | 4.93 | 5 | 0.19 | 0.26 | 0.35 | 0.2 | 0.3 | 0.3 |
| MeCN | 70.0 | 5.57 | 5 | 0.21 | 0.36 | 0.50 | 0.2 | 0.4 | 0.5 |
| MeCN | 70.0 | 6.22 | 5 | 0.18 | 0.32 | 0.43 | 0.2 | 0.3 | 0.4 |
| MeCN | 70.0 | 6.86 | 5 | 0.29 | 0.51 | 0.72 | 0.3 | 0.5 | 0.7 |
| MeCN | 70.0 | 7.46 | 5 | 0.28 | 0.56 | 0.78 | 0.9 | 0.7 | 1.6 |
| MeCN | 0.0 | 7.50 | 5 | 2.2 | >4.0 | >4.0 | | | |
| MeCN | 0.0 | 8.00 | 5 | 2.5 | 3.8 | >4.0 | 2.5 | 3.8 | >4.0 |
| MeCN | 0.0 | 8.50 | 5 | 3.5 | >4.0 | >4.0 | 3.5 | >4.0 | >4.0 |
| MeCN | 0.0 | 9.00 | 5 | 3.2 | 3.9 | >4.0 | 3.2 | 3.9 | >4.0 |
| MeCN | 0.0 | 9.50 | 5 | 3.3 | >4.0 | >4.0 | 3.3 | >4.0 | >4.0 |
| MeCN | 0.0 | 10.00 | 5 | 3.0 | >4.0 | >4.0 | 3.0 | >4.0 | >4.0 |
| MeCN | 0.0 | 10.50 | 5 | 3.3 | >4.0 | >4.0 | 3.3 | >4.0 | >4.0 |
| MeCN | 0.0 | 11.00 | 5 | 2.9 | >4.0 | >4.0 | 2.9 | >4.0 | >4.0 |
| MeCN | 6.4 | 7.50 | 5 | 2.7 | >4.0 | >4.0 | | | |
| MeCN | 6.4 | 8.00 | 5 | 3.1 | >4.0 | >4.0 | 3.1 | >4.0 | >4.0 |
| MeCN | 6.4 | 8.50 | 5 | 3.2 | >4.0 | >4.0 | 3.2 | >4.0 | >4.0 |
| MeCN | 6.4 | 9.00 | 5 | 3.1 | >4.0 | >4.0 | 3.1 | >4.0 | >4.0 |
| MeCN | 6.4 | 9.50 | 5 | 2.9 | >4.0 | >4.0 | 2.9 | >4.0 | >4.0 |
| MeCN | 6.4 | 10.00 | 5 | 3.1 | >4.0 | >4.0 | 3.1 | >4.0 | >4.0 |
| MeCN | 6.4 | 10.50 | 5 | 3.7 | >4.0 | >4.0 | 3.7 | >4.0 | >4.0 |
| MeCN | 6.4 | 11.00 | 5 | 2.9 | >4.0 | >4.0 | 2.9 | >4.0 | >4.0 |
| MeCN | 12.7 | 7.50 | 5 | 2.9 | >4.0 | 4.0 | | | |
| MeCN | 12.7 | 8.00 | 5 | 2.9 | >4.0 | >4.0 | 2.9 | >4.0 | >4.0 |
| MeCN | 12.7 | 8.50 | 5 | 3.1 | >4.0 | >4.0 | 3.1 | >4.0 | >4.0 |
| MeCN | 12.7 | 9.00 | 5 | 2.4 | >4.0 | 3.7 | 2.4 | >4.0 | 3.7 |
| MeCN | 12.7 | 9.50 | 5 | 2.6 | >4.0 | >4.0 | 2.6 | >4.0 | >4.0 |
| MeCN | 12.7 | 10.00 | 5 | 2.5 | >4.0 | >4.0 | 2.5 | >4.0 | >4.0 |
| MeCN | 12.7 | 10.50 | 5 | 3.3 | >4.0 | >4.0 | 3.3 | >4.0 | >4.0 |
| MeCN | 12.7 | 11.00 | 5 | 2.8 | >4.0 | >4.0 | 2.8 | >4.0 | >4.0 |
| MeCN | 19.1 | 7.50 | 5 | 1.9 | >4.0 | 3.2 | | | |
| MeCN | 19.1 | 8.00 | 5 | 1.7 | >4.0 | >4.0 | 1.7 | >4.0 | >4.0 |
| MeCN | 19.1 | 8.50 | 5 | 1.9 | >4.0 | >4.0 | 1.9 | >4.0 | >4.0 |
| MeCN | 19.1 | 9.00 | 5 | 1.7 | >4.0 | >4.0 | 1.7 | >4.0 | >4.0 |
| MeCN | 19.1 | 9.50 | 5 | 1.9 | >4.0 | >4.0 | 1.9 | >4.0 | >4.0 |
| MeCN | 19.1 | 10.00 | 5 | 1.7 | >4.0 | >4.0 | 1.7 | >4.0 | >4.0 |
| MeCN | 19.1 | 10.50 | 5 | 1.9 | >4.0 | >4.0 | 1.9 | >4.0 | >4.0 |
| MeCN | 19.1 | 11.00 | 5 | 1.8 | 3.9 | >4.0 | 1.8 | 3.9 | >4.0 |
| MeCN | 25.5 | 7.50 | 5 | 1.7 | >4.0 | 2.4 | | | |
| MeCN | 25.5 | 8.00 | 5 | 1.2 | >4.0 | 4.0 | 1.2 | >4.0 | 4.0 |
| MeCN | 25.5 | 8.50 | 5 | 1.0 | >4.0 | >4.0 | 1.0 | >4.0 | >4.0 |
| MeCN | 25.5 | 9.00 | 5 | 1.0 | 3.5 | 4.0 | 1.0 | 3.5 | 4.0 |
| MeCN | 25.5 | 9.50 | 5 | 3.2 | >4.0 | >4.0 | 3.2 | >4.0 | >4.0 |
| MeCN | 25.5 | 10.00 | 5 | 0.8 | 3.2 | >4.0 | 0.8 | 3.2 | >4.0 |
| MeCN | 25.5 | 10.50 | 5 | 1.1 | >4.0 | >4.0 | 1.1 | >4.0 | >4.0 |
| MeCN | 25.5 | 11.00 | 5 | 2.6 | >4.0 | >4.0 | 2.6 | >4.0 | >4.0 |
| MeCN | 31.8 | 7.50 | 5 | 0.9 | 3.1 | 2.2 | | | |
| MeCN | 31.8 | 8.00 | 5 | 0.8 | 3.2 | 3.2 | 0.8 | 3.2 | 3.2 |
| MeCN | 31.8 | 8.50 | 5 | 0.9 | >4.0 | >4.0 | 0.9 | >4.0 | >4.0 |
| MeCN | 31.8 | 9.00 | 5 | 0.5 | 3.4 | >4.0 | 0.5 | 3.4 | >4.0 |
| MeCN | 31.8 | 9.50 | 5 | 0.7 | >4.0 | >4.0 | 0.7 | >4.0 | >4.0 |
| MeCN | 31.8 | 10.00 | 5 | 0.8 | >4.0 | >4.0 | 0.8 | >4.0 | >4.0 |
| MeCN | 31.8 | 10.50 | 5 | 1.1 | >4.0 | >4.0 | 1.1 | >4.0 | >4.0 |
| MeCN | 31.8 | 11.00 | 5 | 0.8 | 3.9 | >4.0 | 0.8 | 3.9 | >4.0 |
| MeCN | 38.2 | 7.50 | 5 | 0.8 | 3.8 | 3.1 | | | |
| MeCN | 38.2 | 8.00 | 5 | 0.7 | 3.0 | 3.7 | 0.7 | 3.0 | 3.7 |
| MeCN | 38.2 | 8.50 | 5 | 0.7 | >4.0 | 3.9 | 0.7 | >4.0 | 3.9 |
| MeCN | 38.2 | 9.00 | 5 | 0.6 | >4.0 | >4.0 | 0.6 | >4.0 | >4.0 |
| MeCN | 38.2 | 9.50 | 5 | 0.7 | >4.0 | >4.0 | 0.7 | >4.0 | >4.0 |
| MeCN | 38.2 | 10.00 | 5 | 0.7 | >4.0 | >4.0 | 0.7 | >4.0 | >4.0 |
| MeCN | 38.2 | 10.50 | 5 | 0.7 | 3.7 | >4.0 | 0.7 | 3.7 | >4.0 |
| MeCN | 38.2 | 11.00 | 5 | 0.8 | >4.0 | 3.2 | 0.8 | >4.0 | 3.2 |
| MeCN | 44.5 | 7.50 | 5 | 0.5 | 2.7 | 3.2 | | | |
| MeCN | 44.5 | 8.00 | 5 | 0.4 | 2.7 | 3.4 | 0.4 | 2.7 | 3.4 |
| MeCN | 44.5 | 8.50 | 5 | 0.8 | >4.0 | 2.0 | 0.8 | >4.0 | 2.0 |
| MeCN | 44.5 | 9.00 | 5 | 0.4 | >4.0 | >4.0 | 0.4 | >4.0 | >4.0 |
| MeCN | 44.5 | 9.50 | 5 | 0.6 | 3.8 | >4.0 | 0.6 | 3.8 | >4.0 |
| MeCN | 44.5 | 10.00 | 5 | 0.5 | >4.0 | >4.0 | 0.5 | >4.0 | >4.0 |
| MeCN | 44.5 | 10.50 | 5 | 0.7 | >4.0 | >4.0 | 0.7 | >4.0 | >4.0 |
| MeCN | 44.5 | 11.00 | 5 | 0.4 | >4.0 | >4.0 | 0.4 | >4.0 | >4.0 |
| MeCN | 50.9 | 7.50 | 5 | 0.6 | 1.8 | 2.5 | | | |
| MeCN | 50.9 | 8.00 | 5 | 0.8 | 3.0 | 3.7 | 0.8 | 3.0 | 3.7 |
| MeCN | 50.9 | 8.50 | 5 | 0.8 | >4.0 | 1.5 | 0.8 | >4.0 | 1.5 |
| MeCN | 50.9 | 9.00 | 5 | 0.6 | 3.9 | 2.4 | 0.6 | 3.9 | 2.4 |
| MeCN | 50.9 | 9.50 | 5 | 0.7 | >4.0 | 3.9 | 0.7 | >4.0 | 3.9 |
| MeCN | 50.9 | 10.00 | 5 | 0.5 | 3.9 | >4.0 | 0.5 | 3.9 | >4.0 |
| MeCN | 50.9 | 10.50 | 5 | 0.7 | >4.0 | 3.9 | 0.7 | >4.0 | 3.9 |
| MeCN | 50.9 | 11.00 | 5 | 0.6 | >4.0 | >4.0 | 0.6 | >4.0 | >4.0 |
| MeCN | 57.3 | 7.50 | 5 | 0.6 | 2.2 | 3.2 | | | |
| MeCN | 57.3 | 8.00 | 5 | 0.7 | 2.4 | 3.7 | 0.7 | 2.4 | 3.7 |
| MeCN | 57.3 | 8.50 | 5 | 0.6 | 3.3 | 2.5 | 0.6 | 3.3 | 2.5 |
| MeCN | 57.3 | 9.00 | 5 | 0.6 | 3.4 | 1.4 | 0.6 | 3.4 | 1.4 |
| MeCN | 57.3 | 9.50 | 5 | 0.5 | >4.0 | 2.5 | 0.5 | >4.0 | 2.5 |
| MeCN | 57.3 | 10.00 | 5 | 0.4 | >4.0 | 1.1 | 0.4 | >4.0 | 1.1 |
| MeCN | 57.3 | 10.50 | 5 | 0.5 | >4.0 | 1.7 | 0.5 | >4.0 | 1.7 |
| MeCN | 57.3 | 11.00 | 5 | 0.5 | >4.0 | 3.6 | 0.5 | >4.0 | 3.6 |
| MeCN | 63.6 | 7.50 | 5 | 0.4 | 1.0 | 2.4 | | | |
| MeCN | 63.6 | 8.00 | 5 | 0.5 | 1.2 | 3.2 | 0.5 | 1.2 | 3.2 |
| MeCN | 63.6 | 8.50 | 5 | 0.5 | 3.1 | 3.1 | 0.5 | 3.1 | 3.1 |
| MeCN | 63.6 | 9.00 | 5 | 0.5 | 1.7 | >4.0 | 0.5 | 1.7 | >4.0 |
| MeCN | 63.6 | 9.50 | 5 | 0.6 | 2.9 | 1.2 | 0.6 | 2.9 | 1.2 |
| MeCN | 63.6 | 10.00 | 5 | 0.4 | 2.9 | 1.0 | 0.4 | 2.9 | 1.0 |
| MeCN | 63.6 | 10.50 | 5 | 0.6 | 3.4 | 1.3 | 0.6 | 3.4 | 1.3 |
| MeCN | 63.6 | 11.00 | 5 | 0.5 | 3.2 | 1.0 | 0.5 | 3.2 | 1.0 |
| MeCN | 70.0 | 7.50 | 5 | 0.3 | 0.7 | 1.6 | | | |
| MeCN | 70.0 | 8.00 | 5 | 0.4 | 1.0 | 2.5 | 0.4 | 1.0 | 2.5 |
| MeCN | 70.0 | 8.50 | 5 | 0.4 | 1.4 | 3.3 | 0.4 | 1.4 | 3.3 |
| MeCN | 70.0 | 9.00 | 5 | 0.4 | 1.5 | >4.0 | 0.4 | 1.5 | >4.0 |
| MeCN | 70.0 | 9.50 | 5 | 0.5 | 1.9 | >4.0 | 0.5 | 1.9 | >4.0 |
| MeCN | 70.0 | 10.00 | 5 | 0.4 | 2.4 | 1.0 | 0.4 | 2.4 | 1.0 |
| MeCN | 70.0 | 10.50 | 5 | 0.6 | 3.1 | 1.2 | 0.6 | 3.1 | 1.2 |
| MeCN | 70.0 | 11.00 | 5 | 0.6 | 3.5 | 1.2 | 0.6 | 3.5 | 1.2 |

Conc.: Concentration.
Temp.: Temperature.
n.d.: not determined.

TABLE 3b
Treatment conditions using Method 1 (evaporation) and results in the
form of GLP-1 peptide concentration at specific incubation time.

| Organic solvent | | | | Peptide concentration [mg/ml] | | |
|---|---|---|---|---|---|---|
| | Conc. | | Temp. | after | after | after |
| Type | [% w/w] | pH | [° C.] | 5 min | 180 min | 24 h |
| MeCN | 0.0 | 3.0 | 5° C. | 0.1 | 0.1 | 0.2 |
| MeCN | 0.0 | 3.6 | 5° C. | 0.0 | 0.1 | 0.1 |
| MeCN | 0.0 | 4.3 | 5° C. | 0.1 | 0.1 | 0.2 |
| MeCN | 0.0 | 4.9 | 5° C. | 0.2 | 0.1 | 0.1 |
| MeCN | 0.0 | 5.6 | 5° C. | 0.1 | 0.1 | 0.1 |
| MeCN | 0.0 | 6.2 | 5° C. | 0.1 | 0.1 | 0.1 |
| MeCN | 0.0 | 6.9 | 5° C. | 0.4 | 2.0 | >4.0 |
| MeCN | 0.0 | 7.5 | 5° C. | 0.6 | 2.6 | >4.0 |
| MeCN | 6.4 | 3.0 | 5° C. | 0.1 | 0.2 | 0.3 |
| MeCN | 6.4 | 3.6 | 5° C. | 0.1 | 0.1 | 0.2 |
| MeCN | 6.4 | 4.3 | 5° C. | 0.1 | 0.1 | 0.2 |
| MeCN | 6.4 | 4.9 | 5° C. | 0.1 | 0.1 | 0.2 |
| MeCN | 6.4 | 5.6 | 5° C. | 0.1 | 0.1 | 0.1 |
| MeCN | 6.4 | 6.2 | 5° C. | 0.1 | 0.2 | 0.2 |
| MeCN | 6.4 | 6.9 | 5° C. | 0.8 | 1.9 | >4.0 |
| MeCN | 6.4 | 7.5 | 5° C. | 1.1 | 2.8 | >4.0 |
| MeCN | 12.7 | 3.0 | 5° C. | 0.2 | 0.2 | 0.3 |
| MeCN | 12.7 | 3.6 | 5° C. | 0.1 | 0.2 | 0.2 |
| MeCN | 12.7 | 4.3 | 5° C. | 0.1 | 0.1 | 0.1 |
| MeCN | 12.7 | 4.9 | 5° C. | 0.1 | 0.1 | 0.1 |
| MeCN | 12.7 | 5.6 | 5° C. | 0.1 | 0.1 | 0.1 |
| MeCN | 12.7 | 6.2 | 5° C. | 0.2 | 0.3 | 0.4 |
| MeCN | 12.7 | 6.9 | 5° C. | 1.0 | 2.3 | >4.0 |
| MeCN | 12.7 | 7.5 | 5° C. | 1.3 | 2.3 | >4.0 |
| MeCN | 19.1 | 3.0 | 5° C. | 0.7 | 1.4 | 1.5 |
| MeCN | 19.1 | 3.6 | 5° C. | 0.1 | 0.2 | 0.2 |
| MeCN | 19.1 | 4.3 | 5° C. | 0.1 | 0.1 | 0.1 |
| MeCN | 19.1 | 4.9 | 5° C. | 0.1 | 0.1 | 0.2 |
| MeCN | 19.1 | 5.6 | 5° C. | 0.2 | 0.1 | 0.2 |
| MeCN | 19.1 | 6.2 | 5° C. | 0.7 | 0.6 | 0.4 |
| MeCN | 19.1 | 6.9 | 5° C. | 2.5 | >4.0 | >4.0 |
| MeCN | 19.1 | 7.5 | 5° C. | 1.4 | 2.9 | >4.0 |
| MeCN | 25.5 | 3.0 | 5° C. | 0.4 | 0.7 | 0.7 |
| MeCN | 25.5 | 3.6 | 5° C. | 0.2 | 0.2 | 0.2 |
| MeCN | 25.5 | 4.3 | 5° C. | 0.3 | 0.2 | 0.1 |
| MeCN | 25.5 | 4.9 | 5° C. | 0.1 | 0.2 | 0.1 |
| MeCN | 25.5 | 5.6 | 5° C. | 0.1 | 0.1 | 0.1 |
| MeCN | 25.5 | 6.2 | 5° C. | 1.6 | 0.9 | 0.2 |
| MeCN | 25.5 | 6.9 | 5° C. | 1.3 | 3.5 | >4.0 |
| MeCN | 25.5 | 7.5 | 5° C. | 1.3 | 3.8 | >4.0 |
| MeCN | 31.8 | 3.0 | 5° C. | 1.0 | 0.7 | 0.5 |
| MeCN | 31.8 | 3.6 | 5° C. | 0.1 | 0.3 | 0.2 |
| MeCN | 31.8 | 4.3 | 5° C. | 0.2 | 0.3 | 0.2 |
| MeCN | 31.8 | 4.9 | 5° C. | 0.1 | 0.2 | 0.1 |
| MeCN | 31.8 | 5.6 | 5° C. | 0.3 | 0.3 | 0.1 |
| MeCN | 31.8 | 6.2 | 5° C. | 1.7 | 1.1 | 0.2 |
| MeCN | 31.8 | 6.9 | 5° C. | 0.9 | 3.0 | 2.7 |
| MeCN | 31.8 | 7.5 | 5° C. | 1.1 | 3.4 | >4.0 |
| MeCN | 38.2 | 3.0 | 5° C. | 1.0 | 1.4 | 0.5 |
| MeCN | 38.2 | 3.6 | 5° C. | 0.2 | 0.5 | 0.5 |
| MeCN | 38.2 | 4.3 | 5° C. | 0.2 | 0.3 | 0.2 |
| MeCN | 38.2 | 4.9 | 5° C. | 0.1 | 0.3 | 0.2 |
| MeCN | 38.2 | 5.6 | 5° C. | 0.6 | 0.6 | 0.2 |
| MeCN | 38.2 | 6.2 | 5° C. | 1.2 | 1.1 | 0.4 |
| MeCN | 38.2 | 6.9 | 5° C. | 1.2 | 2.6 | 1.6 |
| MeCN | 38.2 | 7.5 | 5° C. | 1.1 | 4.0 | >4.0 |
| MeCN | 44.5 | 3.0 | 5° C. | 0.4 | 1.4 | 0.7 |
| MeCN | 44.5 | 3.6 | 5° C. | 0.2 | 0.6 | 0.5 |
| MeCN | 44.5 | 4.3 | 5° C. | 0.2 | 0.3 | 0.3 |
| MeCN | 44.5 | 4.9 | 5° C. | 0.2 | 0.3 | 0.3 |
| MeCN | 44.5 | 5.6 | 5° C. | 0.2 | 0.6 | 0.3 |
| MeCN | 44.5 | 6.2 | 5° C. | 1.1 | 1.4 | 1.1 |
| MeCN | 44.5 | 6.9 | 5° C. | 1.4 | 2.0 | 1.9 |
| MeCN | 44.5 | 7.5 | 5° C. | 1.7 | >4.0 | >4.0 |
| MeCN | 50.9 | 3.0 | 5° C. | 0.8 | 1.6 | 1.7 |
| MeCN | 50.9 | 3.6 | 5° C. | 0.2 | 0.5 | 0.7 |
| MeCN | 50.9 | 4.3 | 5° C. | 0.2 | 0.3 | 0.4 |
| MeCN | 50.9 | 4.9 | 5° C. | 0.3 | 0.4 | 0.3 |
| MeCN | 50.9 | 5.6 | 5° C. | 0.4 | 0.7 | 0.6 |
| MeCN | 50.9 | 6.2 | 5° C. | 0.6 | 0.9 | 0.9 |
| MeCN | 50.9 | 6.9 | 5° C. | 0.6 | 1.4 | 1.7 |
| MeCN | 50.9 | 7.5 | 5° C. | 1.0 | 3.0 | >4.0 |
| MeCN | 57.3 | 3.0 | 5° C. | 0.7 | 1.2 | 1.5 |
| MeCN | 57.3 | 3.6 | 5° C. | 0.2 | 0.5 | 0.7 |
| MeCN | 57.3 | 4.3 | 5° C. | 0.2 | 0.4 | 0.5 |
| MeCN | 57.3 | 4.9 | 5° C. | 0.2 | 0.4 | 0.4 |
| MeCN | 57.3 | 5.6 | 5° C. | 0.3 | 0.6 | 0.7 |
| MeCN | 57.3 | 6.2 | 5° C. | 0.6 | 0.7 | 0.7 |
| MeCN | 57.3 | 6.9 | 5° C. | 0.3 | 0.6 | 1.1 |
| MeCN | 57.3 | 7.5 | 5° C. | 1.6 | 2.3 | 2.9 |
| MeCN | 63.6 | 3.0 | 5° C. | 0.4 | 0.7 | 1.2 |
| MeCN | 63.6 | 3.6 | 5° C. | 0.2 | 0.4 | 0.7 |
| MeCN | 63.6 | 4.3 | 5° C. | 0.2 | 0.4 | 0.6 |
| MeCN | 63.6 | 4.9 | 5° C. | 0.2 | 0.4 | 0.5 |
| MeCN | 63.6 | 5.6 | 5° C. | 0.3 | 0.4 | 0.6 |
| MeCN | 63.6 | 6.2 | 5° C. | 0.2 | 0.4 | 0.6 |
| MeCN | 63.6 | 6.9 | 5° C. | 0.2 | 0.4 | 0.6 |
| MeCN | 63.6 | 7.5 | 5° C. | 0.4 | 0.5 | 1.6 |
| MeCN | 70.0 | 3.0 | 5° C. | 0.4 | 0.5 | 1.0 |
| MeCN | 70.0 | 3.6 | 5° C. | 0.2 | 0.4 | 0.6 |
| MeCN | 70.0 | 4.3 | 5° C. | 0.2 | 0.3 | 0.5 |
| MeCN | 70.0 | 4.9 | 5° C. | 0.2 | 0.3 | 0.5 |
| MeCN | 70.0 | 5.6 | 5° C. | 0.2 | 0.3 | 0.4 |
| MeCN | 70.0 | 6.2 | 5° C. | 0.2 | 0.3 | 0.5 |
| MeCN | 70.0 | 6.9 | 5° C. | 0.2 | 0.3 | 0.5 |
| MeCN | 70.0 | 7.5 | 5° C. | 0.3 | 0.4 | 0.8 |
| MeCN | 0.0 | 8.0 | 5° C. | 0.5 | 2.1 | >4.0 |
| MeCN | 0.0 | 8.5 | 5° C. | 0.3 | 2.9 | >4.0 |
| MeCN | 0.0 | 9.0 | 5° C. | 0.3 | 2.8 | >4.0 |
| MeCN | 0.0 | 9.5 | 5° C. | 0.4 | 2.6 | >4.0 |
| MeCN | 0.0 | 10.0 | 5° C. | 0.4 | 3.1 | >4.0 |
| MeCN | 0.0 | 10.5 | 5° C. | 0.5 | 2.9 | >4.0 |
| MeCN | 0.0 | 11.0 | 5° C. | 0.2 | 1.6 | >4.0 |
| MeCN | 6.4 | 8.0 | 5° C. | 0.8 | 2.2 | >4.0 |
| MeCN | 6.4 | 8.5 | 5° C. | 0.8 | 2.6 | >4.0 |
| MeCN | 6.4 | 9.0 | 5° C. | 1.0 | 2.9 | >4.0 |
| MeCN | 6.4 | 9.5 | 5° C. | 0.6 | 2.5 | >4.0 |
| MeCN | 6.4 | 10.0 | 5° C. | 1.0 | 3.1 | >4.0 |
| MeCN | 6.4 | 10.5 | 5° C. | 0.7 | 2.6 | >4.0 |
| MeCN | 6.4 | 11.0 | 5° C. | 0.7 | 1.7 | >4.0 |
| MeCN | 12.7 | 8.0 | 5° C. | 1.3 | 2.2 | >4.0 |
| MeCN | 12.7 | 8.5 | 5° C. | 0.8 | 2.1 | >4.0 |
| MeCN | 12.7 | 9.0 | 5° C. | 0.8 | 1.8 | >4.0 |
| MeCN | 12.7 | 9.5 | 5° C. | 0.9 | 1.9 | >4.0 |
| MeCN | 12.7 | 10.0 | 5° C. | 0.8 | 2.3 | >4.0 |
| MeCN | 12.7 | 10.5 | 5° C. | 1.1 | 2.1 | >4.0 |
| MeCN | 12.7 | 11.0 | 5° C. | 0.6 | 1.5 | >4.0 |
| MeCN | 19.1 | 8.0 | 5° C. | 1.0 | 2.7 | >4.0 |
| MeCN | 19.1 | 8.5 | 5° C. | 1.2 | 2.5 | >4.0 |
| MeCN | 19.1 | 9.0 | 5° C. | 0.9 | 2.3 | >4.0 |
| MeCN | 19.1 | 9.5 | 5° C. | 1.1 | 3.3 | >4.0 |
| MeCN | 19.1 | 10.0 | 5° C. | 1.4 | 3.8 | >4.0 |
| MeCN | 19.1 | 10.5 | 5° C. | 1.4 | >4.0 | >4.0 |
| MeCN | 19.1 | 11.0 | 5° C. | 1.0 | 2.6 | >4.0 |
| MeCN | 25.5 | 8.0 | 5° C. | 1.1 | 3.2 | >4.0 |
| MeCN | 25.5 | 8.5 | 5° C. | 1.4 | 3.5 | >4.0 |
| MeCN | 25.5 | 9.0 | 5° C. | 0.5 | >4.0 | >4.0 |
| MeCN | 25.5 | 9.5 | 5° C. | 1.2 | >4.0 | >4.0 |
| MeCN | 25.5 | 10.0 | 5° C. | 1.4 | 3.4 | >4.0 |
| MeCN | 25.5 | 10.5 | 5° C. | 1.5 | 3.8 | >4.0 |
| MeCN | 25.5 | 11.0 | 5° C. | 1.0 | 3.1 | >4.0 |
| MeCN | 31.8 | 8.0 | 5° C. | 0.9 | 2.7 | >4.0 |
| MeCN | 31.8 | 8.5 | 5° C. | 2.1 | 3.6 | >4.0 |
| MeCN | 31.8 | 9.0 | 5° C. | 0.8 | 2.7 | >4.0 |
| MeCN | 31.8 | 9.5 | 5° C. | 0.9 | 3.3 | >4.0 |
| MeCN | 31.8 | 10.0 | 5° C. | 1.9 | >4.0 | 2.8 |
| MeCN | 31.8 | 10.5 | 5° C. | 1.3 | 3.9 | 3.6 |
| MeCN | 31.8 | 11.0 | 5° C. | 0.8 | 3.0 | >4.0 |
| MeCN | 38.2 | 8.0 | 5° C. | 0.8 | 3.2 | >4.0 |
| MeCN | 38.2 | 8.5 | 5° C. | 0.8 | 2.9 | >4.0 |
| MeCN | 38.2 | 9.0 | 5° C. | 0.4 | 2.0 | >4.0 |
| MeCN | 38.2 | 9.5 | 5° C. | 1.1 | 3.9 | 4.0 |
| MeCN | 38.2 | 10.0 | 5° C. | 1.5 | >4.0 | >4.0 |
| MeCN | 38.2 | 10.5 | 5° C. | 1.2 | 3.2 | 2.1 |
| MeCN | 38.2 | 11.0 | 5° C. | 0.6 | 2.7 | >4.0 |
| MeCN | 44.5 | 8.0 | 5° C. | 0.7 | 2.8 | >4.0 |
| MeCN | 44.5 | 8.5 | 5° C. | 1.1 | 3.3 | >4.0 |
| MeCN | 44.5 | 9.0 | 5° C. | 1.7 | 2.7 | >4.0 |
| MeCN | 44.5 | 9.5 | 5° C. | 1.1 | 3.7 | 1.3 |
| MeCN | 44.5 | 10.0 | 5° C. | 1.3 | 3.7 | >4.0 |
| MeCN | 44.5 | 10.5 | 5° C. | 0.9 | 2.9 | >4.0 |
| MeCN | 44.5 | 11.0 | 5° C. | 0.8 | 2.9 | >4.0 |
| MeCN | 50.9 | 8.0 | 5° C. | 0.6 | 2.2 | >4.0 |
| MeCN | 50.9 | 8.5 | 5° C. | 0.6 | 2.6 | >4.0 |
| MeCN | 50.9 | 9.0 | 5° C. | 0.6 | 2.0 | >4.0 |
| MeCN | 50.9 | 9.5 | 5° C. | 1.0 | 2.4 | >4.0 |
| MeCN | 50.9 | 10.0 | 5° C. | 1.5 | 2.6 | >4.0 |
| MeCN | 50.9 | 10.5 | 5° C. | 0.9 | 2.1 | 3.8 |
| MeCN | 50.9 | 11.0 | 5° C. | 0.7 | 2.2 | >4.0 |
| MeCN | 57.3 | 8.0 | 5° C. | 0.5 | 1.6 | 3.4 |
| MeCN | 57.3 | 8.5 | 5° C. | 0.5 | 1.6 | 3.7 |
| MeCN | 57.3 | 9.0 | 5° C. | 0.5 | 1.9 | >4.0 |
| MeCN | 57.3 | 9.5 | 5° C. | 1.0 | 2.0 | 4.0 |
| MeCN | 57.3 | 10.0 | 5° C. | 1.4 | 1.6 | 1.8 |
| MeCN | 57.3 | 10.5 | 5° C. | 1.1 | 1.1 | 1.6 |
| MeCN | 57.3 | 11.0 | 5° C. | 0.9 | 1.4 | 2.6 |
| MeCN | 63.6 | 8.0 | 5° C. | 0.3 | 0.6 | 2.0 |
| MeCN | 63.6 | 8.5 | 5° C. | 0.6 | 0.8 | 2.1 |
| MeCN | 63.6 | 9.0 | 5° C. | 1.4 | 0.9 | 2.6 |
| MeCN | 63.6 | 9.5 | 5° C. | 0.2 | 0.5 | 1.4 |
| MeCN | 63.6 | 10.0 | 5° C. | 0.2 | 0.3 | 0.7 |
| MeCN | 63.6 | 10.5 | 5° C. | 0.2 | 0.3 | 0.6 |
| MeCN | 63.6 | 11.0 | 5° C. | 0.5 | 0.6 | 1.3 |
| MeCN | 70.0 | 8.0 | 5° C. | 0.3 | 0.4 | 0.6 |
| MeCN | 70.0 | 8.5 | 5° C. | 0.2 | 0.4 | 0.7 |
| MeCN | 70.0 | 9.0 | 5° C. | 0.2 | 0.4 | 0.9 |
| MeCN | 70.0 | 9.5 | 5° C. | 0.2 | 0.3 | 0.5 |
| MeCN | 70.0 | 10.0 | 5° C. | 0.2 | 0.3 | 0.4 |
| MeCN | 70.0 | 10.5 | 5° C. | 0.2 | 0.2 | 0.4 |
| MeCN | 70.0 | 11.0 | 5° C. | 0.2 | 0.2 | 0.5 |
| MeCN | 0.0 | 3.0 | RT | 0.1 | 0.1 | 0.1 |
| MeCN | 0.0 | 3.6 | RT | 0.1 | 0.1 | 0.1 |
| MeCN | 0.0 | 4.3 | RT | 0.1 | 0.1 | 0.0 |
| MeCN | 0.0 | 4.9 | RT | 0.3 | 0.1 | n.d. |
| MeCN | 0.0 | 5.6 | RT | 0.2 | 0.1 | 0.0 |
| MeCN | 0.0 | 6.2 | RT | 0.1 | 0.4 | 0.5 |
| MeCN | 0.0 | 6.9 | RT | 0.6 | 3.1 | >4.0 |
| MeCN | 0.0 | 7.5 | RT | 0.8 | 3.0 | >4.0 |
| MeCN | 6.4 | 3.0 | RT | 0.2 | 0.1 | 0.1 |
| MeCN | 6.4 | 3.6 | RT | 0.1 | 0.1 | 0.1 |
| MeCN | 6.4 | 4.3 | RT | 0.1 | 0.1 | 0.1 |
| MeCN | 6.4 | 4.9 | RT | 0.1 | 0.1 | 0.1 |
| MeCN | 6.4 | 5.6 | RT | 0.1 | 0.1 | 0.1 |
| MeCN | 6.4 | 6.2 | RT | 0.4 | 0.9 | 1.4 |
| MeCN | 6.4 | 6.9 | RT | 0.9 | 2.7 | >4.0 |
| MeCN | 6.4 | 7.5 | RT | 1.1 | 3.1 | >4.0 |
| MeCN | 12.7 | 3.0 | RT | 0.3 | 0.1 | 0.1 |
| MeCN | 12.7 | 3.6 | RT | 0.1 | 0.1 | 0.1 |
| MeCN | 12.7 | 4.3 | RT | 0.1 | 0.1 | 0.1 |
| MeCN | 12.7 | 4.9 | RT | 0.1 | 0.1 | 0.0 |
| MeCN | 12.7 | 5.6 | RT | 0.2 | 0.2 | 0.1 |
| MeCN | 12.7 | 6.2 | RT | 0.5 | 1.2 | 1.3 |
| MeCN | 12.7 | 6.9 | RT | 1.2 | 2.6 | >4.0 |
| MeCN | 12.7 | 7.5 | RT | 1.4 | 3.6 | >4.0 |
| MeCN | 19.1 | 3.0 | RT | 1.0 | 1.6 | 1.6 |
| MeCN | 19.1 | 3.6 | RT | 0.1 | 0.1 | 0.0 |
| MeCN | 19.1 | 4.3 | RT | 0.1 | 0.1 | 0.0 |
| MeCN | 19.1 | 4.9 | RT | 0.1 | 0.1 | 0.0 |
| MeCN | 19.1 | 5.6 | RT | 0.6 | 0.1 | 0.1 |
| MeCN | 19.1 | 6.2 | RT | 0.8 | 0.8 | 0.8 |
| MeCN | 19.1 | 6.9 | RT | 1.2 | >4.0 | >4.0 |
| MeCN | 19.1 | 7.5 | RT | 1.6 | >4.0 | >4.0 |
| MeCN | 25.5 | 3.0 | RT | 0.5 | 0.4 | 0.4 |
| MeCN | 25.5 | 3.6 | RT | 0.2 | 0.1 | 0.1 |
| MeCN | 25.5 | 4.3 | RT | 0.1 | 0.1 | 0.1 |
| MeCN | 25.5 | 4.9 | RT | 0.1 | 0.1 | 0.0 |
| MeCN | 25.5 | 5.6 | RT | 0.6 | 0.2 | 0.1 |
| MeCN | 25.5 | 6.2 | RT | 1.0 | 0.6 | 0.2 |
| MeCN | 25.5 | 6.9 | RT | 1.2 | 3.5 | 1.4 |
| MeCN | 25.5 | 7.5 | RT | 1.4 | >4.0 | >4.0 |
| MeCN | 31.8 | 3.0 | RT | 0.4 | 0.2 | 0.2 |
| MeCN | 31.8 | 3.6 | RT | 0.4 | 0.2 | 0.1 |
| MeCN | 31.8 | 4.3 | RT | 0.2 | 0.1 | 0.1 |
| MeCN | 31.8 | 4.9 | RT | 0.1 | 0.1 | 0.0 |
| MeCN | 31.8 | 5.6 | RT | 0.8 | 0.3 | 0.1 |
| MeCN | 31.8 | 6.2 | RT | 1.2 | 0.6 | 0.1 |
| MeCN | 31.8 | 6.9 | RT | 1.0 | 3.0 | 1.6 |
| MeCN | 31.8 | 7.5 | RT | 1.1 | >4.0 | >4.0 |
| MeCN | 38.2 | 3.0 | RT | 0.4 | 0.5 | 0.1 |
| MeCN | 38.2 | 3.6 | RT | 0.2 | 0.3 | 0.0 |
| MeCN | 38.2 | 4.3 | RT | 0.3 | 0.2 | 0.1 |
| MeCN | 38.2 | 4.9 | RT | 0.2 | 0.2 | 0.0 |
| MeCN | 38.2 | 5.6 | RT | 0.7 | 0.5 | 0.1 |
| MeCN | 38.2 | 6.2 | RT | 1.1 | 0.9 | 0.2 |
| MeCN | 38.2 | 6.9 | RT | 1.5 | 2.3 | 0.6 |
| MeCN | 38.2 | 7.5 | RT | 1.0 | >4.0 | >4.0 |
| MeCN | 44.5 | 3.0 | RT | 0.5 | 0.9 | 0.1 |
| MeCN | 44.5 | 3.6 | RT | 0.3 | 0.5 | 0.0 |
| MeCN | 44.5 | 4.3 | RT | 0.2 | 0.2 | 0.1 |
| MeCN | 44.5 | 4.9 | RT | 0.2 | 0.2 | 0.1 |
| MeCN | 44.5 | 5.6 | RT | 0.5 | 0.7 | 0.1 |
| MeCN | 44.5 | 6.2 | RT | 1.4 | 1.2 | 0.7 |
| MeCN | 44.5 | 6.9 | RT | 0.9 | 1.9 | 1.3 |
| MeCN | 44.5 | 7.5 | RT | 1.3 | >4.0 | >4.0 |
| MeCN | 50.9 | 3.0 | RT | 0.6 | 1.2 | 0.1 |
| MeCN | 50.9 | 3.6 | RT | 0.3 | 0.4 | 0.4 |
| MeCN | 50.9 | 4.3 | RT | 0.2 | 0.3 | 0.2 |
| MeCN | 50.9 | 4.9 | RT | 0.2 | 0.3 | 0.2 |
| MeCN | 50.9 | 5.6 | RT | 0.5 | 0.7 | 0.4 |
| MeCN | 50.9 | 6.2 | RT | 0.7 | 0.8 | 0.7 |
| MeCN | 50.9 | 6.9 | RT | 0.6 | 1.5 | 0.0 |
| MeCN | 50.9 | 7.5 | RT | 1.0 | >4.0 | >4.0 |
| MeCN | 57.3 | 3.0 | RT | 0.4 | 1.0 | 1.3 |
| MeCN | 57.3 | 3.6 | RT | 0.2 | 0.4 | 0.4 |
| MeCN | 57.3 | 4.3 | RT | 0.2 | 0.4 | 0.3 |
| MeCN | 57.3 | 4.9 | RT | 0.2 | 0.4 | 0.2 |
| MeCN | 57.3 | 5.6 | RT | 0.3 | 0.6 | 0.5 |
| MeCN | 57.3 | 6.2 | RT | 0.5 | 0.5 | 0.6 |
| MeCN | 57.3 | 6.9 | RT | 0.4 | 0.8 | 0.9 |
| MeCN | 57.3 | 7.5 | RT | 0.8 | 3.0 | >4.0 |
| MeCN | 63.6 | 3.0 | RT | 0.3 | 0.8 | 1.1 |
| MeCN | 63.6 | 3.6 | RT | 0.2 | 0.3 | 0.4 |
| MeCN | 63.6 | 4.3 | RT | 0.2 | 0.3 | 0.4 |
| MeCN | 63.6 | 4.9 | RT | 0.2 | 0.4 | 0.3 |
| MeCN | 63.6 | 5.6 | RT | 0.3 | 0.4 | 0.5 |
| MeCN | 63.6 | 6.2 | RT | 0.3 | 0.4 | 0.4 |
| MeCN | 63.6 | 6.9 | RT | 0.2 | 0.4 | 0.5 |
| MeCN | 63.6 | 7.5 | RT | 0.5 | 1.6 | 3.1 |
| MeCN | 70.0 | 3.0 | RT | 0.4 | 0.5 | 0.8 |
| MeCN | 70.0 | 3.6 | RT | 0.2 | 0.3 | 0.4 |
| MeCN | 70.0 | 4.3 | RT | 0.2 | 0.3 | 0.3 |
| MeCN | 70.0 | 4.9 | RT | 0.2 | 0.3 | 0.3 |
| MeCN | 70.0 | 5.6 | RT | 0.2 | 0.2 | 0.3 |
| MeCN | 70.0 | 6.2 | RT | 0.2 | 0.3 | 0.5 |
| MeCN | 70.0 | 6.9 | RT | 0.2 | 0.2 | 0.4 |
| MeCN | 70.0 | 7.5 | RT | 0.3 | 0.8 | 1.9 |
| MeCN | 0.0 | 8.0 | RT | 0.6 | 2.5 | >4.0 |
| MeCN | 0.0 | 8.5 | RT | 0.6 | 3.5 | >4.0 |
| MeCN | 0.0 | 9.0 | RT | 0.7 | 2.9 | >4.0 |
| MeCN | 0.0 | 9.5 | RT | 0.8 | 2.8 | >4.0 |
| MeCN | 0.0 | 10.0 | RT | 0.9 | 3.4 | >4.0 |
| MeCN | 0.0 | 10.5 | RT | 0.8 | 2.9 | >4.0 |
| MeCN | 0.0 | 11.0 | RT | 0.7 | 2.2 | >4.0 |
| MeCN | 6.4 | 8.0 | RT | 1.0 | 2.2 | >4.0 |
| MeCN | 6.4 | 8.5 | RT | 0.9 | 2.7 | >4.0 |
| MeCN | 6.4 | 9.0 | RT | 0.9 | 2.5 | >4.0 |
| MeCN | 6.4 | 9.5 | RT | 0.9 | 2.4 | >4.0 |
| MeCN | 6.4 | 10.0 | RT | 1.0 | 3.1 | >4.0 |
| MeCN | 6.4 | 10.5 | RT | 0.9 | 2.8 | >4.0 |
| MeCN | 6.4 | 11.0 | RT | 0.8 | 2.3 | >4.0 |
| MeCN | 12.7 | 8.0 | RT | 3.2 | 3.8 | >4.0 |
| MeCN | 12.7 | 8.5 | RT | 0.9 | 2.6 | >4.0 |
| MeCN | 12.7 | 9.0 | RT | 1.0 | 2.6 | >4.0 |
| MeCN | 12.7 | 9.5 | RT | 1.1 | 2.9 | >4.0 |
| MeCN | 12.7 | 10.0 | RT | 1.0 | 3.3 | >4.0 |
| MeCN | 12.7 | 10.5 | RT | 1.2 | 3.3 | >4.0 |
| MeCN | 12.7 | 11.0 | RT | 1.0 | 2.9 | >4.0 |
| MeCN | 19.1 | 8.0 | RT | 1.6 | 3.9 | >4.0 |
| MeCN | 19.1 | 8.5 | RT | 1.2 | 3.7 | >4.0 |
| MeCN | 19.1 | 9.0 | RT | 1.0 | 3.7 | >4.0 |
| MeCN | 19.1 | 9.5 | RT | 1.7 | >4.0 | >4.0 |
| MeCN | 19.1 | 10.0 | RT | 1.3 | >4.0 | >4.0 |
| MeCN | 19.1 | 10.5 | RT | 1.6 | >4.0 | >4.0 |
| MeCN | 19.1 | 11.0 | RT | 1.3 | 3.6 | >4.0 |
| MeCN | 25.5 | 8.0 | RT | 1.1 | 3.9 | >4.0 |
| MeCN | 25.5 | 8.5 | RT | 1.0 | >4.0 | >4.0 |
| MeCN | 25.5 | 9.0 | RT | 0.5 | 3.5 | >4.0 |
| MeCN | 25.5 | 9.5 | RT | 1.5 | >4.0 | >4.0 |
| MeCN | 25.5 | 10.0 | RT | 1.6 | >4.0 | >4.0 |
| MeCN | 25.5 | 10.5 | RT | 1.7 | >4.0 | >4.0 |
| MeCN | 25.5 | 11.0 | RT | 1.2 | >4.0 | >4.0 |
| MeCN | 31.8 | 8.0 | RT | 0.9 | >4.0 | >4.0 |
| MeCN | 31.8 | 8.5 | RT | 1.1 | >4.0 | >4.0 |
| MeCN | 31.8 | 9.0 | RT | 1.2 | 3.8 | >4.0 |
| MeCN | 31.8 | 9.5 | RT | 1.3 | >4.0 | >4.0 |
| MeCN | 31.8 | 10.0 | RT | 1.6 | >4.0 | >4.0 |
| MeCN | 31.8 | 10.5 | RT | 1.4 | >4.0 | >4.0 |
| MeCN | 31.8 | 11.0 | RT | 1.1 | >4.0 | >4.0 |
| MeCN | 38.2 | 8.0 | RT | 1.4 | >4.0 | >4.0 |
| MeCN | 38.2 | 8.5 | RT | 1.7 | >4.0 | >4.0 |
| MeCN | 38.2 | 9.0 | RT | 1.1 | >4.0 | >4.0 |
| MeCN | 38.2 | 9.5 | RT | 1.0 | >4.0 | >4.0 |
| MeCN | 38.2 | 10.0 | RT | 1.9 | >4.0 | >4.0 |
| MeCN | 38.2 | 10.5 | RT | 1.6 | >4.0 | 3.8 |
| MeCN | 38.2 | 11.0 | RT | 1.2 | >4.0 | 3.4 |
| MeCN | 44.5 | 8.0 | RT | 1.0 | >4.0 | >4.0 |
| MeCN | 44.5 | 8.5 | RT | 1.1 | 3.9 | >4.0 |
| MeCN | 44.5 | 9.0 | RT | 0.8 | 3.5 | >4.0 |
| MeCN | 44.5 | 9.5 | RT | 1.0 | >4.0 | >4.0 |
| MeCN | 44.5 | 10.0 | RT | 2.0 | >4.0 | 3.7 |
| MeCN | 44.5 | 10.5 | RT | 1.1 | >4.0 | 4.0 |
| MeCN | 44.5 | 11.0 | RT | 1.3 | >4.0 | >4.0 |
| MeCN | 50.9 | 8.0 | RT | 0.7 | 3.8 | >4.0 |
| MeCN | 50.9 | 8.5 | RT | 1.0 | 3.7 | >4.0 |
| MeCN | 50.9 | 9.0 | RT | 0.7 | 2.8 | >4.0 |
| MeCN | 50.9 | 9.5 | RT | 1.6 | >4.0 | 4.0 |
| MeCN | 50.9 | 10.0 | RT | 1.9 | >4.0 | 3.9 |
| MeCN | 50.9 | 10.5 | RT | 1.0 | 3.7 | >4.0 |
| MeCN | 50.9 | 11.0 | RT | 0.8 | >4.0 | >4.0 |
| MeCN | 57.3 | 8.0 | RT | 0.6 | 2.8 | >4.0 |
| MeCN | 57.3 | 8.5 | RT | 0.5 | 2.9 | >4.0 |
| MeCN | 57.3 | 9.0 | RT | 0.4 | 2.2 | >4.0 |
| MeCN | 57.3 | 9.5 | RT | 0.7 | 3.0 | >4.0 |
| MeCN | 57.3 | 10.0 | RT | 1.0 | 2.0 | >4.0 |
| MeCN | 57.3 | 10.5 | RT | 0.8 | 1.6 | >4.0 |
| MeCN | 57.3 | 11.0 | RT | 0.7 | 2.7 | >4.0 |
| MeCN | 63.6 | 8.0 | RT | 0.3 | 1.2 | 3.3 |
| MeCN | 63.6 | 8.5 | RT | 0.3 | 1.3 | 2.9 |
| MeCN | 63.6 | 9.0 | RT | 0.3 | 1.5 | >4.0 |
| MeCN | 63.6 | 9.5 | RT | 0.3 | 1.6 | >4.0 |
| MeCN | 63.6 | 10.0 | RT | 0.4 | 0.6 | 1.6 |
| MeCN | 63.6 | 10.5 | RT | 0.4 | 0.6 | 1.8 |
| MeCN | 63.6 | 11.0 | RT | 0.4 | 1.6 | 0.9 |
| MeCN | 70.0 | 8.0 | RT | 0.2 | 0.9 | 1.9 |
| MeCN | 70.0 | 8.5 | RT | 0.2 | 0.5 | 1.6 |
| MeCN | 70.0 | 9.0 | RT | 0.2 | 1.0 | 2.5 |
| MeCN | 70.0 | 9.5 | RT | 0.2 | 0.4 | 1.6 |
| MeCN | 70.0 | 10.0 | RT | 0.2 | 0.3 | 1.0 |
| MeCN | 70.0 | 10.5 | RT | 0.2 | 0.3 | 1.1 |
| MeCN | 70.0 | 11.0 | RT | 0.2 | 0.8 | 2.3 |
| MeCN | 0.0 | 3.0 | 70° C. | 1.0 | 1.5 | n.d. |
| MeCN | 0.0 | 3.6 | 70° C. | 0.0 | 0.2 | n.d. |
| MeCN | 0.0 | 4.3 | 70° C. | 0.1 | 0.1 | n.d. |
| MeCN | 0.0 | 4.9 | 70° C. | 0.9 | 0.0 | n.d. |
| MeCN | 0.0 | 5.6 | 70° C. | 0.3 | 1.7 | n.d. |
| MeCN | 0.0 | 6.2 | 70° C. | 0.8 | >4.0 | n.d. |
| MeCN | 0.0 | 6.9 | 70° C. | 2.0 | >4.0 | n.d. |
| MeCN | 0.0 | 7.5 | 70° C. | 1.8 | >4.0 | n.d. |
| MeCN | 6.4 | 3.0 | 70° C. | 2.2 | >4.0 | n.d. |
| MeCN | 6.4 | 3.6 | 70° C. | 0.4 | 0.3 | n.d. |
| MeCN | 6.4 | 4.3 | 70° C. | 0.1 | 0.1 | n.d. |
| MeCN | 6.4 | 4.9 | 70° C. | 0.3 | 0.1 | n.d. |
| MeCN | 6.4 | 5.6 | 70° C. | 0.7 | 1.0 | n.d. |
| MeCN | 6.4 | 6.2 | 70° C. | 2.4 | >4.0 | n.d. |
| MeCN | 6.4 | 6.9 | 70° C. | 3.0 | >4.0 | n.d. |
| MeCN | 6.4 | 7.5 | 70° C. | 2.5 | >4.0 | n.d. |
| MeCN | 12.7 | 3.0 | 70° C. | 3.5 | >4.0 | n.d. |
| MeCN | 12.7 | 3.6 | 70° C. | 0.3 | 0.1 | n.d. |
| MeCN | 12.7 | 4.3 | 70° C. | 0.1 | 0.0 | n.d. |
| MeCN | 12.7 | 4.9 | 70° C. | 0.1 | 0.1 | n.d. |
| MeCN | 12.7 | 5.6 | 70° C. | 0.5 | 0.1 | n.d. |
| MeCN | 12.7 | 6.2 | 70° C. | 1.8 | 1.3 | n.d. |
| MeCN | 12.7 | 6.9 | 70° C. | 3.2 | >4.0 | n.d. |
| MeCN | 12.7 | 7.5 | 70° C. | 3.7 | >4.0 | n.d. |
| MeCN | 19.1 | 3.0 | 70° C. | 1.8 | 1.1 | n.d. |
| MeCN | 19.1 | 3.6 | 70° C. | 0.1 | 0.0 | n.d. |
| MeCN | 19.1 | 4.3 | 70° C. | 0.1 | 0.1 | n.d. |
| MeCN | 19.1 | 4.9 | 70° C. | 0.1 | 0.1 | n.d. |
| MeCN | 19.1 | 5.6 | 70° C. | 0.2 | 0.1 | n.d. |
| MeCN | 19.1 | 6.2 | 70° C. | 1.3 | 0.7 | n.d. |
| MeCN | 19.1 | 6.9 | 70° C. | >4.0 | >4.0 | n.d. |
| MeCN | 19.1 | 7.5 | 70° C. | >4.0 | >4.0 | n.d. |
| MeCN | 25.5 | 3.0 | 70° C. | 0.6 | 0.9 | n.d. |
| MeCN | 25.5 | 3.6 | 70° C. | 0.1 | 0.1 | n.d. |
| MeCN | 25.5 | 4.3 | 70° C. | 0.1 | 0.1 | n.d. |
| MeCN | 25.5 | 4.9 | 70° C. | 0.1 | 0.1 | n.d. |
| MeCN | 25.5 | 5.6 | 70° C. | 0.2 | 0.1 | n.d. |
| MeCN | 25.5 | 6.2 | 70° C. | 0.9 | 0.3 | n.d. |
| MeCN | 25.5 | 6.9 | 70° C. | 3.8 | 3.5 | n.d. |
| MeCN | 25.5 | 7.5 | 70° C. | >4.0 | >4.0 | n.d. |
| MeCN | 31.8 | 3.0 | 70° C. | 0.7 | 1.3 | n.d. |
| MeCN | 31.8 | 3.6 | 70° C. | 0.2 | 0.2 | n.d. |
| MeCN | 31.8 | 4.3 | 70° C. | 0.2 | 0.2 | n.d. |
| MeCN | 31.8 | 4.9 | 70° C. | 0.2 | 0.1 | n.d. |
| MeCN | 31.8 | 5.6 | 70° C. | 0.7 | 0.2 | n.d. |
| MeCN | 31.8 | 6.2 | 70° C. | 1.4 | 0.8 | n.d. |
| MeCN | 31.8 | 6.9 | 70° C. | 3.3 | 3.5 | n.d. |
| MeCN | 31.8 | 7.5 | 70° C. | >4.0 | >4.0 | n.d. |
| MeCN | 38.2 | 3.0 | 70° C. | 0.7 | 1.1 | n.d. |
| MeCN | 38.2 | 3.6 | 70° C. | 0.4 | 0.6 | n.d. |
| MeCN | 38.2 | 4.3 | 70° C. | 0.4 | 0.3 | n.d. |
| MeCN | 38.2 | 4.9 | 70° C. | 0.3 | 0.3 | n.d. |
| MeCN | 38.2 | 5.6 | 70° C. | 1.1 | 0.8 | n.d. |
| MeCN | 38.2 | 6.2 | 70° C. | 1.7 | 1.4 | n.d. |
| MeCN | 38.2 | 6.9 | 70° C. | 2.7 | 3.8 | n.d. |
| MeCN | 38.2 | 7.5 | 70° C. | >4.0 | >4.0 | n.d. |
| MeCN | 44.5 | 3.0 | 70° C. | 1.2 | 2.0 | n.d. |
| MeCN | 44.5 | 3.6 | 70° C. | 0.5 | 0.8 | n.d. |
| MeCN | 44.5 | 4.3 | 70° C. | 0.5 | 0.6 | n.d. |
| MeCN | 44.5 | 4.9 | 70° C. | 0.4 | 0.5 | n.d. |
| MeCN | 44.5 | 5.6 | 70° C. | 1.1 | 1.4 | n.d. |
| MeCN | 44.5 | 6.2 | 70° C. | 1.8 | 2.2 | n.d. |
| MeCN | 44.5 | 6.9 | 70° C. | 2.7 | >4.0 | n.d. |
| MeCN | 44.5 | 7.5 | 70° C. | >4.0 | >4.0 | n.d. |
| MeCN | 50.9 | 3.0 | 70° C. | 1.0 | n.d. | n.d. |
| MeCN | 50.9 | 3.6 | 70° C. | 0.5 | 0.9 | n.d. |
| MeCN | 50.9 | 4.3 | 70° C. | 0.6 | 0.9 | n.d. |
| MeCN | 50.9 | 4.9 | 70° C. | 0.5 | 0.9 | n.d. |
| MeCN | 50.9 | 5.6 | 70° C. | 1.0 | 1.5 | n.d. |
| MeCN | 50.9 | 6.2 | 70° C. | 1.0 | 2.0 | n.d. |
| MeCN | 50.9 | 6.9 | 70° C. | 2.2 | 3.6 | n.d. |
| MeCN | 50.9 | 7.5 | 70° C. | >4.0 | >4.0 | n.d. |
| MeCN | 57.3 | 3.0 | 70° C. | 0.9 | 2.6 | n.d. |
| MeCN | 57.3 | 3.6 | 70° C. | 0.5 | n.d. | n.d. |
| MeCN | 57.3 | 4.3 | 70° C. | 0.5 | 1.3 | n.d. |
| MeCN | 57.3 | 4.9 | 70° C. | 0.6 | 1.3 | n.d. |
| MeCN | 57.3 | 5.6 | 70° C. | 0.9 | 1.8 | n.d. |
| MeCN | 57.3 | 6.2 | 70° C. | 0.9 | 2.0 | n.d. |
| MeCN | 57.3 | 6.9 | 70° C. | 0.9 | 2.7 | n.d. |
| MeCN | 57.3 | 7.5 | 70° C. | 3.3 | >4.0 | n.d. |
| MeCN | 63.6 | 3.0 | 70° C. | 0.9 | 2.8 | n.d. |
| MeCN | 63.6 | 3.6 | 70° C. | 0.6 | n.d. | n.d. |
| MeCN | 63.6 | 4.3 | 70° C. | 0.5 | 1.8 | n.d. |
| MeCN | 63.6 | 4.9 | 70° C. | 0.5 | 1.7 | n.d. |
| MeCN | 63.6 | 5.6 | 70° C. | 0.7 | 1.7 | n.d. |
| MeCN | 63.6 | 6.2 | 70° C. | 0.5 | 2.2 | n.d. |
| MeCN | 63.6 | 6.9 | 70° C. | 0.5 | 2.4 | n.d. |
| MeCN | 63.6 | 7.5 | 70° C. | 1.9 | >4.0 | n.d. |
| MeCN | 70.0 | 3.0 | 70° C. | 0.6 | 2.9 | n.d. |
| MeCN | 70.0 | 3.6 | 70° C. | 0.4 | 2.5 | n.d. |
| MeCN | 70.0 | 4.3 | 70° C. | 0.5 | 2.4 | n.d. |
| MeCN | 70.0 | 4.9 | 70° C. | 0.5 | 2.4 | n.d. |
| MeCN | 70.0 | 5.6 | 70° C. | 0.5 | 2.6 | n.d. |
| MeCN | 70.0 | 6.2 | 70° C. | 0.5 | 3.7 | n.d. |
| MeCN | 70.0 | 6.9 | 70° C. | 0.5 | 3.6 | n.d. |
| MeCN | 70.0 | 7.5 | 70° C. | 0.7 | 3.3 | n.d. |
| MeCN | 0.0 | 8.0 | 70° C. | 2.2 | >4.0 | n.d. |
| MeCN | 0.0 | 8.5 | 70° C. | 1.9 | >4.0 | n.d. |
| MeCN | 0.0 | 9.0 | 70° C. | 2.1 | >4.0 | n.d. |
| MeCN | 0.0 | 9.5 | 70° C. | 2.3 | >4.0 | n.d. |
| MeCN | 0.0 | 10.0 | 70° C. | 2.2 | >4.0 | n.d. |
| MeCN | 0.0 | 10.5 | 70° C. | 2.6 | >4.0 | n.d. |
| MeCN | 0.0 | 11.0 | 70° C. | 2.0 | >4.0 | n.d. |
| MeCN | 6.4 | 8.0 | 70° C. | 2.3 | >4.0 | n.d. |
| MeCN | 6.4 | 8.5 | 70° C. | 2.5 | >4.0 | n.d. |
| MeCN | 6.4 | 9.0 | 70° C. | 2.9 | >4.0 | n.d. |
| MeCN | 6.4 | 9.5 | 70° C. | 3.2 | >4.0 | n.d. |
| MeCN | 6.4 | 10.0 | 70° C. | 3.1 | >4.0 | n.d. |
| MeCN | 6.4 | 10.5 | 70° C. | 3.3 | >4.0 | n.d. |
| MeCN | 6.4 | 11.0 | 70° C. | 2.5 | >4.0 | n.d. |
| MeCN | 12.7 | 8.0 | 70° C. | 3.6 | >4.0 | n.d. |
| MeCN | 12.7 | 8.5 | 70° C. | 3.5 | >4.0 | n.d. |
| MeCN | 12.7 | 9.0 | 70° C. | 3.5 | >4.0 | n.d. |
| MeCN | 12.7 | 9.5 | 70° C. | >4.0 | >4.0 | n.d. |
| MeCN | 12.7 | 10.0 | 70° C. | >4.0 | >4.0 | n.d. |
| MeCN | 12.7 | 10.5 | 70° C. | >4.0 | >4.0 | n.d. |
| MeCN | 12.7 | 11.0 | 70° C. | 3.3 | >4.0 | n.d. |
| MeCN | 19.1 | 8.0 | 70° C. | >4.0 | >4.0 | n.d. |
| MeCN | 19.1 | 8.5 | 70° C. | >4.0 | >4.0 | n.d. |
| MeCN | 19.1 | 9.0 | 70° C. | >4.0 | >4.0 | n.d. |
| MeCN | 19.1 | 9.5 | 70° C. | >4.0 | >4.0 | n.d. |
| MeCN | 19.1 | 10.0 | 70° C. | >4.0 | >4.0 | n.d. |
| MeCN | 19.1 | 10.5 | 70° C. | >4.0 | >4.0 | n.d. |
| MeCN | 19.1 | 11.0 | 70° C. | 3.8 | >4.0 | n.d. |
| MeCN | 25.5 | 8.0 | 70° C. | >4.0 | >4.0 | n.d. |
| MeCN | 25.5 | 8.5 | 70° C. | 4.0 | >4.0 | n.d. |
| MeCN | 25.5 | 9.0 | 70° C. | 3.7 | >4.0 | n.d. |
| MeCN | 25.5 | 9.5 | 70° C. | >4.0 | >4.0 | n.d. |
| MeCN | 25.5 | 10.0 | 70° C. | >4.0 | >4.0 | n.d. |
| MeCN | 25.5 | 10.5 | 70° C. | >4.0 | >4.0 | n.d. |
| MeCN | 25.5 | 11.0 | 70° C. | >4.0 | >4.0 | n.d. |
| MeCN | 31.8 | 8.0 | 70° C. | 4.0 | >4.0 | n.d. |
| MeCN | 31.8 | 8.5 | 70° C. | >4.0 | >4.0 | n.d. |
| MeCN | 31.8 | 9.0 | 70° C. | >4.0 | >4.0 | n.d. |
| MeCN | 31.8 | 9.5 | 70° C. | 3.8 | >4.0 | n.d. |
| MeCN | 31.8 | 10.0 | 70° C. | >4.0 | >4.0 | n.d. |
| MeCN | 31.8 | 10.5 | 70° C. | >4.0 | >4.0 | n.d. |
| MeCN | 31.8 | 11.0 | 70° C. | 3.7 | >4.0 | n.d. |
| MeCN | 38.2 | 8.0 | 70° C. | 3.9 | >4.0 | n.d. |
| MeCN | 38.2 | 8.5 | 70° C. | >4.0 | >4.0 | n.d. |
| MeCN | 38.2 | 9.0 | 70° C. | 2.8 | >4.0 | n.d. |
| MeCN | 38.2 | 9.5 | 70° C. | 4.0 | >4.0 | n.d. |
| MeCN | 38.2 | 10.0 | 70° C. | >4.0 | >4.0 | n.d. |
| MeCN | 38.2 | 10.5 | 70° C. | 3.3 | >4.0 | n.d. |
| MeCN | 38.2 | 11.0 | 70° C. | 3.8 | >4.0 | n.d. |
| MeCN | 44.5 | 8.0 | 70° C. | 3.5 | >4.0 | n.d. |
| MeCN | 44.5 | 8.5 | 70° C. | 3.6 | >4.0 | n.d. |
| MeCN | 44.5 | 9.0 | 70° C. | 2.2 | >4.0 | n.d. |
| MeCN | 44.5 | 9.5 | 70° C. | >4.0 | >4.0 | n.d. |
| MeCN | 44.5 | 10.0 | 70° C. | >4.0 | >4.0 | n.d. |
| MeCN | 44.5 | 10.5 | 70° C. | >4.0 | >4.0 | n.d. |
| MeCN | 44.5 | 11.0 | 70° C. | 3.9 | >4.0 | n.d. |
| MeCN | 50.9 | 8.0 | 70° C. | 3.7 | >4.0 | n.d. |
| MeCN | 50.9 | 8.5 | 70° C. | 2.9 | >4.0 | n.d. |
| MeCN | 50.9 | 9.0 | 70° C. | 1.8 | >4.0 | n.d. |
| MeCN | 50.9 | 9.5 | 70° C. | >4.0 | >4.0 | n.d. |
| MeCN | 50.9 | 10.0 | 70° C. | >4.0 | >4.0 | n.d. |
| MeCN | 50.9 | 10.5 | 70° C. | 3.9 | >4.0 | n.d. |
| MeCN | 50.9 | 11.0 | 70° C. | 2.6 | >4.0 | n.d. |
| MeCN | 57.3 | 8.0 | 70° C. | 3.4 | >4.0 | n.d. |
| MeCN | 57.3 | 8.5 | 70° C. | 3.7 | >4.0 | n.d. |
| MeCN | 57.3 | 9.0 | 70° C. | 1.6 | >4.0 | n.d. |
| MeCN | 57.3 | 9.5 | 70° C. | 4.0 | >4.0 | n.d. |
| MeCN | 57.3 | 10.0 | 70° C. | 2.5 | >4.0 | n.d. |
| MeCN | 57.3 | 10.5 | 70° C. | 2.6 | >4.0 | n.d. |
| MeCN | 57.3 | 11.0 | 70° C. | 3.6 | >4.0 | n.d. |
| MeCN | 63.6 | 8.0 | 70° C. | 2.0 | >4.0 | n.d. |
| MeCN | 63.6 | 8.5 | 70° C. | 2.2 | >4.0 | n.d. |
| MeCN | 63.6 | 9.0 | 70° C. | 1.4 | >4.0 | n.d. |
| MeCN | 63.6 | 9.5 | 70° C. | 2.9 | >4.0 | n.d. |
| MeCN | 63.6 | 10.0 | 70° C. | 1.0 | >4.0 | n.d. |
| MeCN | 63.6 | 10.5 | 70° C. | 0.9 | >4.0 | n.d. |
| MeCN | 63.6 | 11.0 | 70° C. | 2.4 | >4.0 | n.d. |
| MeCN | 70.0 | 8.0 | 70° C. | 1.2 | 3.8 | n.d. |
| MeCN | 70.0 | 8.5 | 70° C. | 0.8 | 3.5 | n.d. |
| MeCN | 70.0 | 9.0 | 70° C. | 0.9 | >4.0 | n.d. |
| MeCN | 70.0 | 9.5 | 70° C. | 1.0 | 3.4 | n.d. |
| MeCN | 70.0 | 10.0 | 70° C. | 0.5 | 2.9 | n.d. |
| MeCN | 70.0 | 10.5 | 70° C. | 0.4 | 3.3 | n.d. |
| MeCN | 70.0 | 11.0 | 70° C. | 0.8 | 3.4 | n.d. |
| DMF | 0.0 | 3.1 | RT | 0.2 | 0.2 | 0.3 |
| DMF | 0.0 | 4.8 | RT | 0.2 | 0.2 | 0.2 |
| DMF | 0.0 | 5.8 | RT | 0.2 | 1.1 | 1.5 |
| DMF | 0.0 | 6.3 | RT | 0.9 | 2.4 | >4.0 |
| DMF | 0.0 | 6.7 | RT | 1.0 | 3.1 | 2.2 |
| DMF | 0.0 | 6.9 | RT | 1.1 | >4.0 | >4.0 |
| DMF | 0.0 | 7.2 | RT | 0.7 | >4.0 | >4.0 |
| DMF | 0.0 | 7.5 | RT | 1.9 | >4.0 | n.d. |
| DMF | 6.4 | 3.1 | RT | 0.2 | 0.3 | 0.8 |
| DMF | 6.4 | 4.8 | RT | 0.2 | 0.4 | 0.2 |
| DMF | 6.4 | 5.8 | RT | 0.3 | 2.9 | 2.1 |
| DMF | 6.4 | 6.3 | RT | 0.9 | 2.5 | >4.0 |
| DMF | 6.4 | 6.7 | RT | 1.2 | 2.5 | >4.0 |
| DMF | 6.4 | 6.9 | RT | 1.1 | 3.2 | >4.0 |
| DMF | 6.4 | 7.2 | RT | 1.1 | 3.0 | >4.0 |
| DMF | 6.4 | 7.5 | RT | 1.8 | >4.0 | n.d. |
| DMF | 12.7 | 3.1 | RT | 0.2 | 0.4 | 1.2 |
| DMF | 12.7 | 4.8 | RT | 0.2 | 0.5 | 0.3 |
| DMF | 12.7 | 5.8 | RT | 0.3 | 3.1 | 2.3 |
| DMF | 12.7 | 6.3 | RT | 0.9 | 2.3 | >4.0 |
| DMF | 12.7 | 6.7 | RT | 1.0 | 2.0 | 3.9 |
| DMF | 12.7 | 6.9 | RT | 1.0 | 3.0 | >4.0 |
| DMF | 12.7 | 7.2 | RT | 0.9 | 2.3 | >4.0 |
| DMF | 12.7 | 7.5 | RT | 1.7 | >4.0 | n.d. |
| DMF | 19.1 | 3.1 | RT | 0.2 | 0.4 | 1.3 |
| DMF | 19.1 | 4.8 | RT | 0.2 | 0.5 | 0.3 |
| DMF | 19.1 | 5.8 | RT | 0.3 | 2.6 | 1.2 |
| DMF | 19.1 | 6.3 | RT | 0.9 | 2.0 | >4.0 |
| DMF | 19.1 | 6.7 | RT | 0.8 | 1.3 | 2.1 |
| DMF | 19.1 | 6.9 | RT | 1.0 | 2.8 | >4.0 |
| DMF | 19.1 | 7.2 | RT | 0.7 | 2.3 | >4.0 |
| DMF | 19.1 | 7.5 | RT | 2.3 | >4.0 | n.d. |
| DMF | 25.5 | 3.1 | RT | 0.2 | 0.3 | 1.0 |
| DMF | 25.5 | 4.8 | RT | 0.2 | 0.3 | 0.2 |
| DMF | 25.5 | 5.8 | RT | 0.4 | 2.1 | 1.3 |
| DMF | 25.5 | 6.3 | RT | 0.7 | 1.5 | >4.0 |
| DMF | 25.5 | 6.7 | RT | 0.7 | 1.8 | 2.7 |
| DMF | 25.5 | 6.9 | RT | 0.9 | 2.2 | >4.0 |
| DMF | 25.5 | 7.2 | RT | 0.4 | 1.7 | >4.0 |
| DMF | 25.5 | 7.5 | RT | 2.7 | >4.0 | n.d. |
| DMF | 31.8 | 3.1 | RT | 0.2 | 0.2 | 0.6 |
| DMF | 31.8 | 4.8 | RT | 0.2 | 0.3 | 0.3 |
| DMF | 31.8 | 5.8 | RT | 0.3 | 1.1 | 0.8 |
| DMF | 31.8 | 6.3 | RT | 0.5 | 0.9 | 2.6 |
| DMF | 31.8 | 6.7 | RT | 0.6 | 1.4 | 2.0 |
| DMF | 31.8 | 6.9 | RT | 0.9 | 1.8 | 2.2 |
| DMF | 31.8 | 7.2 | RT | 0.8 | 1.7 | >4.0 |
| DMF | 31.8 | 7.5 | RT | 2.6 | >4.0 | n.d. |
| DMF | 38.2 | 3.1 | RT | 0.2 | 0.2 | 0.4 |
| DMF | 38.2 | 4.8 | RT | 0.2 | 0.3 | 0.3 |
| DMF | 38.2 | 5.8 | RT | 0.5 | 0.6 | 0.7 |
| DMF | 38.2 | 6.3 | RT | 0.5 | 0.6 | 1.5 |
| DMF | 38.2 | 6.7 | RT | 0.7 | 0.8 | 1.1 |
| DMF | 38.2 | 6.9 | RT | 0.5 | 0.6 | 0.8 |
| DMF | 38.2 | 7.2 | RT | 0.6 | 0.7 | 1.6 |
| DMF | 38.2 | 7.5 | RT | 1.5 | n.d. | n.d. |
| DMF | 44.5 | 3.1 | RT | 0.2 | 0.2 | 0.3 |
| DMF | 44.5 | 4.8 | RT | 0.2 | 0.2 | 0.2 |
| DMF | 44.5 | 5.8 | RT | 0.4 | 0.4 | 0.5 |
| DMF | 44.5 | 6.3 | RT | 0.3 | 0.3 | 0.6 |
| DMF | 44.5 | 6.7 | RT | 0.4 | 0.4 | 0.6 |
| DMF | 44.5 | 6.9 | RT | 0.4 | 0.6 | 0.7 |
| DMF | 44.5 | 7.2 | RT | 0.4 | 0.6 | 1.6 |
| DMF | 44.5 | 7.5 | RT | 1.8 | >4.0 | n.d. |
| DMF | 50.9 | 3.1 | RT | 0.2 | 0.2 | 0.3 |
| DMF | 50.9 | 4.8 | RT | 0.2 | 0.2 | 0.2 |
| DMF | 50.9 | 5.8 | RT | 0.3 | 0.3 | 0.2 |
| DMF | 50.9 | 6.3 | RT | 0.3 | 0.3 | 0.5 |
| DMF | 50.9 | 6.7 | RT | 0.3 | 0.3 | 0.4 |
| DMF | 50.9 | 6.9 | RT | 0.3 | 0.4 | 0.5 |
| DMF | 50.9 | 7.2 | RT | 0.3 | 0.4 | 0.7 |
| DMF | 50.9 | 7.5 | RT | 1.0 | >4.0 | n.d. |
| DMF | 57.3 | 3.1 | RT | 0.2 | 0.2 | 0.3 |
| DMF | 57.3 | 4.8 | RT | 0.2 | 0.2 | 0.2 |
| DMF | 57.3 | 5.8 | RT | 0.3 | 0.3 | 0.2 |
| DMF | 57.3 | 6.3 | RT | 0.2 | 0.3 | 0.4 |
| DMF | 57.3 | 6.7 | RT | 0.3 | 0.3 | 0.3 |
| DMF | 57.3 | 6.9 | RT | 0.7 | 0.4 | 0.4 |
| DMF | 57.3 | 7.2 | RT | 0.3 | 0.4 | 0.5 |
| DMF | 57.3 | 7.5 | RT | 1.6 | >4.0 | n.d. |
| DMF | 63.6 | 3.1 | RT | 0.3 | 0.3 | 0.4 |
| DMF | 63.6 | 4.8 | RT | 0.2 | 0.2 | 0.2 |
| DMF | 63.6 | 5.8 | RT | 0.2 | 0.3 | 0.3 |
| DMF | 63.6 | 6.3 | RT | 0.2 | 0.3 | 0.3 |
| DMF | 63.6 | 6.7 | RT | 0.3 | 0.3 | 0.3 |
| DMF | 63.6 | 6.9 | RT | 0.3 | 0.4 | 0.3 |
| DMF | 63.6 | 7.2 | RT | 0.4 | 0.5 | 0.5 |
| DMF | 63.6 | 7.5 | RT | 3.4 | >4.0 | n.d. |
| DMF | 70.0 | 3.1 | RT | 0.3 | 0.3 | 0.4 |
| DMF | 70.0 | 4.8 | RT | 0.2 | 0.2 | 0.3 |
| DMF | 70.0 | 5.8 | RT | 0.2 | 0.3 | 0.4 |
| DMF | 70.0 | 6.3 | RT | 0.2 | 0.3 | 0.3 |
| DMF | 70.0 | 6.7 | RT | 0.5 | 0.3 | 0.3 |
| DMF | 70.0 | 6.9 | RT | 0.5 | 0.4 | 0.4 |
| DMF | 70.0 | 7.2 | RT | 0.3 | 0.4 | 0.4 |
| DMF | 70.0 | 7.5 | RT | 1.5 | >4.0 | n.d. |
| DMF | 0.0 | 8.0 | RT | 2.2 | >4.0 | n.d. |
| DMF | 0.0 | 8.5 | RT | 2.0 | >4.0 | n.d. |
| DMF | 0.0 | 9.0 | RT | 2.7 | >4.0 | n.d. |
| DMF | 0.0 | 9.5 | RT | 2.7 | >4.0 | n.d. |
| DMF | 0.0 | 10.0 | RT | 2.6 | >4.0 | n.d. |
| DMF | 0.0 | 10.5 | RT | 2.9 | >4.0 | n.d. |
| DMF | 0.0 | 11.0 | RT | 2.4 | >4.0 | n.d. |
| DMF | 6.4 | 8.0 | RT | 1.6 | >4.0 | n.d. |
| DMF | 6.4 | 8.5 | RT | 2.1 | >4.0 | n.d. |
| DMF | 6.4 | 9.0 | RT | 2.0 | >4.0 | n.d. |
| DMF | 6.4 | 9.5 | RT | 2.2 | >4.0 | n.d. |
| DMF | 6.4 | 10.0 | RT | 2.3 | >4.0 | n.d. |
| DMF | 6.4 | 10.5 | RT | 2.4 | >4.0 | n.d. |
| DMF | 6.4 | 11.0 | RT | 1.9 | >4.0 | n.d. |
| DMF | 12.7 | 8.0 | RT | 1.4 | >4.0 | n.d. |
| DMF | 12.7 | 8.5 | RT | 1.9 | >4.0 | n.d. |
| DMF | 12.7 | 9.0 | RT | 1.9 | >4.0 | n.d. |
| DMF | 12.7 | 9.5 | RT | 2.3 | >4.0 | n.d. |
| DMF | 12.7 | 10.0 | RT | 2.3 | >4.0 | n.d. |
| DMF | 12.7 | 10.5 | RT | 2.7 | >4.0 | n.d. |
| DMF | 12.7 | 11.0 | RT | 1.7 | >4.0 | n.d. |
| DMF | 19.1 | 8.0 | RT | 1.4 | >4.0 | n.d. |
| DMF | 19.1 | 8.5 | RT | 1.8 | >4.0 | n.d. |
| DMF | 19.1 | 9.0 | RT | 1.9 | >4.0 | n.d. |
| DMF | 19.1 | 9.5 | RT | 2.6 | >4.0 | n.d. |
| DMF | 19.1 | 10.0 | RT | 2.4 | >4.0 | n.d. |
| DMF | 19.1 | 10.5 | RT | 2.3 | >4.0 | n.d. |
| DMF | 19.1 | 11.0 | RT | 1.5 | >4.0 | n.d. |
| DMF | 25.5 | 8.0 | RT | 1.7 | >4.0 | n.d. |
| DMF | 25.5 | 8.5 | RT | 1.9 | >4.0 | n.d. |
| DMF | 25.5 | 9.0 | RT | 1.3 | >4.0 | n.d. |
| DMF | 25.5 | 9.5 | RT | 3.1 | >4.0 | n.d. |
| DMF | 25.5 | 10.0 | RT | 3.2 | >4.0 | n.d. |
| DMF | 25.5 | 10.5 | RT | 2.9 | >4.0 | n.d. |
| DMF | 25.5 | 11.0 | RT | 2.0 | >4.0 | n.d. |
| DMF | 31.8 | 8.0 | RT | 0.9 | 3.6 | n.d. |
| DMF | 31.8 | 8.5 | RT | 1.5 | >4.0 | n.d. |
| DMF | 31.8 | 9.0 | RT | 0.7 | >4.0 | n.d. |
| DMF | 31.8 | 9.5 | RT | 2.5 | >4.0 | n.d. |
| DMF | 31.8 | 10.0 | RT | 3.3 | >4.0 | n.d. |
| DMF | 31.8 | 10.5 | RT | 2.3 | >4.0 | n.d. |
| DMF | 31.8 | 11.0 | RT | 1.8 | 3.7 | n.d. |
| DMF | 38.2 | 8.0 | RT | 0.7 | >4.0 | n.d. |
| DMF | 38.2 | 8.5 | RT | 1.5 | >4.0 | n.d. |
| DMF | 38.2 | 9.0 | RT | 1.1 | >4.0 | n.d. |
| DMF | 38.2 | 9.5 | RT | >4.0 | >4.0 | n.d. |
| DMF | 38.2 | 10.0 | RT | 3.2 | >4.0 | n.d. |
| DMF | 38.2 | 10.5 | RT | 2.9 | >4.0 | n.d. |
| DMF | 38.2 | 11.0 | RT | 0.6 | >4.0 | n.d. |
| DMF | 44.5 | 8.0 | RT | 1.0 | >4.0 | n.d. |
| DMF | 44.5 | 8.5 | RT | 1.8 | >4.0 | n.d. |
| DMF | 44.5 | 9.0 | RT | 3.6 | 3.8 | n.d. |
| DMF | 44.5 | 9.5 | RT | 3.3 | >4.0 | n.d. |
| DMF | 44.5 | 10.0 | RT | >4.0 | >4.0 | n.d. |
| DMF | 44.5 | 10.5 | RT | 3.0 | >4.0 | n.d. |
| DMF | 44.5 | 11.0 | RT | 3.1 | 3.8 | n.d. |
| DMF | 50.9 | 8.0 | RT | 1.0 | >4.0 | n.d. |
| DMF | 50.9 | 8.5 | RT | 2.1 | >4.0 | n.d. |
| DMF | 50.9 | 9.0 | RT | 3.2 | >4.0 | n.d. |
| DMF | 50.9 | 9.5 | RT | 2.6 | >4.0 | n.d. |
| DMF | 50.9 | 10.0 | RT | 3.8 | >4.0 | n.d. |
| DMF | 50.9 | 10.5 | RT | 4.0 | >4.0 | n.d. |
| DMF | 50.9 | 11.0 | RT | 1.7 | >4.0 | n.d. |
| DMF | 57.3 | 8.0 | RT | 1.3 | >4.0 | n.d. |
| DMF | 57.3 | 8.5 | RT | 3.3 | >4.0 | n.d. |
| DMF | 57.3 | 9.0 | RT | 2.7 | >4.0 | n.d. |
| DMF | 57.3 | 9.5 | RT | 2.5 | >4.0 | n.d. |
| DMF | 57.3 | 10.0 | RT | 3.1 | >4.0 | n.d. |
| DMF | 57.3 | 10.5 | RT | 3.7 | >4.0 | n.d. |
| DMF | 57.3 | 11.0 | RT | 2.1 | >4.0 | n.d. |
| DMF | 63.6 | 8.0 | RT | 1.6 | >4.0 | n.d. |
| DMF | 63.6 | 8.5 | RT | 3.8 | >4.0 | n.d. |
| DMF | 63.6 | 9.0 | RT | 3.1 | >4.0 | n.d. |
| DMF | 63.6 | 9.5 | RT | 2.9 | >4.0 | n.d. |
| DMF | 63.6 | 10.0 | RT | 3.7 | >4.0 | n.d. |
| DMF | 63.6 | 10.5 | RT | 3.5 | >4.0 | n.d. |
| DMF | 63.6 | 11.0 | RT | 1.3 | >4.0 | n.d. |
| DMF | 70.0 | 8.0 | RT | 1.2 | >4.0 | n.d. |
| DMF | 70.0 | 8.5 | RT | 2.9 | >4.0 | n.d. |
| DMF | 70.0 | 9.0 | RT | 2.6 | >4.0 | n.d. |
| DMF | 70.0 | 9.5 | RT | 3.2 | >4.0 | n.d. |
| DMF | 70.0 | 10.0 | RT | 3.4 | >4.0 | n.d. |
| DMF | 70.0 | 10.5 | RT | 3.5 | >4.0 | n.d. |
| DMF | 70.0 | 11.0 | RT | 1.1 | >4.0 | n.d. |

Conc.: Concentration.
Temp.: Temperature.
n.d.: not determined.

The results in Table 3a and 3b show that at certain conditions the presence of organic solvent increases the amount of dissolved GLP-1 peptide.

The terms “organic solvent” or “modifier” as used herein means organic solvent.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for dissolving undissolved and/or insoluble GLP-1 peptides into active, soluble GLP-1 peptides in solutions or suspensions comprising water and one or more organic solvents; comprising the steps of:
(a) obtaining a first composition comprising undissolved and/or insoluble GLP-1 peptide and the organic solvent acetonitrile or DMF, wherein said first composition is in the form of a solution or a suspension; and
(b) incubating said first composition at a temperature in the range of 2-85° C., and
when said organic solvent is DMF then said incubating is carried out for a period of at least 20 minutes and (i) at a pH of less than 5.0 and at a concentration of said DMF in the range of 5-28%(w/w), or (ii) at a pH in the range of 5.0-5.5 and at a concentration of said DMF in the range of 0-35%(w/w); or
when said organic solvent is acetonitrile then said incubating is carried out for a period of at least 2 minutes and (i) at a pH of less than 6.0 and at a concentration of said acetonitrile of at least 30%(w/w), (ii) at a pH of less than 3.5 and at a concentration of said acetonitrile of at least 14%(w/w), or (iii) if said temperature is at least 50° C. then (1) at a pH in the range of 5.5-6.0 and at a concentration of said acetonitrile of less than 10%(w/w), or (2) at a pH of less than 3.5; and
wherein the undissolved and/or insoluble GLP-1 peptide is liraglutide.

2. The method according to claim 1, wherein said method provides an increase in dissolved liraglutide by at least 0.5 mg/ml after an incubation period of 3 hours, optionally determined as described in Assay (I) herein.

3. The method according to claim 1, wherein the temperature of said incubation in step (b) is selected from the group consisting of 2-40° C., 40-85° C. or 10-50° C.

4. The method according to claim 1, wherein said first composition is in the form of a solution or a suspension.

5. The method according to claim 1, wherein said organic solvent is DMF, and in step (b)(i) the concentration of said DMF is in the range of 7-23%(w/w).

6. The method according to claim 5, wherein said period is at least 1 hour.

7. The method according to claim 1 wherein said organic solvent is DMF, and in step (b)(i)
a. said pH is less than 3.8,
b. the concentration of said DMF is in the range of 9-25%(w/w), and
c. said period is at least 8 hours.

8. The method according to claim 1 wherein said organic solvent is DMF, and in step (b) if the incubation time is at least 8 hours, then the incubation is carried out (i) at a pH of less than 5.0 and at a concentration of said DMF in the range of 2-40%(w/w); or (ii) at a pH in the range of 5.0-6.0 and at a concentration of said DMF in the range of 0-40%(w/w).

9. The method according to claim 1, wherein said organic solvent is DMF, and in step (b)(ii) the concentration of said DMF is up to 20%(w/w) or up to 19%(w/w).

10. The method according to claim 1, wherein said organic solvent is acetonitrile, and in step (b)(ii) the concentration of said acetonitrile is at least 20%(w/w) or at least 55%(w/w).

11. The method according to claim 1, wherein said organic solvent is acetonitrile, and in step (b)(ii) said pH is less than 3.4 or less than 3.2.

12. The method according to claim 1, wherein said organic solvent is acetonitrile, and in step (b)(ii) said period is up to 16 hours or in the range of 5 min to 20 hours.

13. The method according to claim 1, wherein said organic solvent is acetonitrile, and in step (b)(iii)(2) the concentration of said acetonitrile is at least 40%(w/w) or at least 45%(w/w).

14. The method according to claim 1, wherein said organic solvent is acetonitrile, and in step (b)(ii) or in step (b)(iii)(2) said pH is less than 3.4 or less than 3.3.

15. The method according to claim 1, wherein said method comprises a step of obtaining a composition comprising substantially no acetonitrile or DMF.
